(19) United States
(12) Patent Application Publication
(10) Pub. No.: US 2023/0200935 A1
(43) Pub. Date: Jun. 29, 2023

(54) POSITIONING ELEMENT FOR POSITIONING AND FIXING A DRILLING TEMPLATE

(71) Applicant: EXOCAD GMBH, Darmstadt (DE)

(72) Inventors: Sebastian RUEHL, Darmstadt (DE); Paul SCHNITZSPAN, Darmstadt (DE)

(21) Appl. No.: 17/925,793

(22) PCT Filed: May 19, 2020
(86) PCT No.: PCT/EP2020/064001

§ 371 Date: Nov. 16, 2022

(51) Int. Cl. A61C 1/08; A61B 34/10; A61C 9/00

(52) U.S. Cl. CPC A61C 1/082; A61B 34/10; A61C 9/0006; A61B 2034/105

(57) ABSTRACT

The invention relates to a method for producing a positioning element for fixing a drilling template on a gingiva of a patient's jaw in a position relative to a jawbone of the jaw. The positioning element is configured to fix the drilling template, by means of pressure, which the patient applies to the positioning element by closing the jaw and an opposing jaw, in the position for making implant holes in the jaw for dental implants.

The invention relates to a method for producing a positioning element for fixing a drilling template on a gingiva of a patient's jaw, to a corresponding positioning element, and to a combination of a corresponding positioning element and a drilling template.

In implantology, the exact positions at which implants are to be placed are planned in advance on the basis of anatomical conditions, in particular of the jaw. In order to be able to make implant holes exactly at the positions planned for the implants, drilling templates are used. These drilling templates define the positions of the implant holes that are to be made, according to what has previously been planned.

However, the positions defined for implant holes by the drilling template depend on the position of the drilling template on the patient's jaw. If a drilling template sits on the soft, yielding gingiva, i.e. the gums, of the jaw, there is a risk that the drilling template will at least partially lack sufficient support. The support provided may therefore be insufficient for precise positioning of the implant holes that are to be made, for example in the submillimeter range. If the drilling template moves, i.e. the actual position thereof on the patient's jaw does not match the position for which the drilling template was configured, the positions defined for the implant holes by the drilling template also no longer match the planned positions.

The object of the invention is to provide methods and means for improved fixing of a drilling template on a gingiva of a patient's jaw.

The object of the invention is respectively achieved by the features of the independent claims. Embodiments of the invention are specified in the dependent claims.

By way of example, a method is provided for producing a first positioning element for fixing a first drilling template on a gingiva of a patient's jaw in a first position relative to a jawbone of the jaw. The first positioning element is configured to fix the first drilling template, by means of pressure, which the patient applies to the first positioning element by closing the jaw and an opposing jaw, in the first position for making first implant holes in the jaw for first dental implants. The first drilling template comprises a first template contact surface for making contact with a surface of the gingiva of the jaw and comprises first through-openings. The first through-openings each define a first implant position for making one of the first implant holes.

The method comprises:
recording first shape data of a first impression of the surface of the gingiva of the jaw, using a bite register arranged between the patient's two jaws, wherein the two jaws are located in a recording position relative to each other, wherein the first impression is located in the first position relative to the jawbone, and wherein the first impression defines the first template contact surface,
recording first structure data of the jawbone while the bite register is arranged between the two jaws in the recording position,
recording position data of the bite register relative to the jawbone while the bite register is arranged between the two jaws in the recording position,
creating a first three-dimensional digital model, using the recorded first shape data, first structure data and position data, wherein the first model comprises the jawbone structure and the surface of the gingiva of the jaw,
defining the first implant positions for making the first implant holes in the jaw, using the first model,
creating a second three-dimensional digital model of the first drilling template and of the first positioning element, using the first model and the defined first implant positions, wherein the shape and dimensions of the first positioning element are determined such that, when pressure is applied to the first positioning element, the two jaws are arranged in the recording position and the first drilling template is arranged in the first position relative to the jawbone,
producing the first positioning element, using the second three-dimensional digital model.

This could have the advantage that a positioning element for a drilling template can be provided which is configured to arrange and to fix a drilling template, in particular a template contact surface, in a predetermined position relative to a patient's jawbone. By way of example, the resulting position of the drilling template or template contact surface reproduces a position of a bite register, in particular of an impression in the bite register, relative to the jawbone. By way of example, the reproduced position of the bite register or of the impression is a position of the bite register or of the impression during the recording of structure data of the jaw or jawbone.

The template contact surface is a surface of the drilling template that comes into contact with the surface of the gingiva of the patient's jaw. By way of example, the shape of the template contact surface is identical to an impression of the gingival surface of the jaw that is recorded using the bite register. By way of example, the shape data of the corresponding impression are used as a specification for the shape of the template contact surface. The purpose of recording the impression may be, for example, to provide a shape specification for the template contact surface. It can thus be ensured, for example, that the template contact surface is a negative of the gingival surface and, for example, sits on the gingival surface with an exact fit.

Owing to the fact that the gingiva yields, there is a risk that unwanted deviations may occur between the actual position of the template contact surface relative to the jawbone and the reference position of the impression of the bite register during the recording of the jawbone structure data. The positioning element may have the advantage that it enables a positioning of the drilling template and/or an orientation of the drilling template in the patient's oral cavity, as a result of which unwanted deviations can be prevented.

Besides the positioning of the drilling template, the positioning element may also have the advantage that, when pressure is applied to the positioning element, the two jaws are arranged in a position relative to each other which corresponds to a recording position. This recording position is the position that the two jaws assumed relative to each other during the recording of the jawbone structure data. This recording position of the jaws results, for example, from arranging the bite register between the jaws during the recording of the jawbone structure data. The recording position may depend, for example, on the shape and dimensions of the bite register. The recording position may also depend, for example, on the pressure that the two jaws apply to the bite register during the recording of the jawbone structure data.

By way of example, the shape and dimensions of the positioning element may be selected such that the position of the jawbones relative to each other corresponds to the recording position. In particular, the shape and dimensions of the positioning element may be selected such that a distance between the jawbones corresponds to a recording distance between the jawbones during the recording of the jawbone structure data. If exactly one drilling template and exactly one positioning element are to be arranged between the patient's jaws, the shape and dimensions of the positioning element may be selected, for example, such that the shape and dimensions of the positioning element in combination with the drilling template correspond to the shape and dimensions of the bite register.

If two drilling templates and exactly one positioning element are to be arranged between the patient's jaws, the shape and dimensions of the positioning element may be selected, for example, such that the shape and dimensions of the positioning element in combination with the two drilling templates correspond to the shape and dimensions of the bite register.

If two drilling templates and two positioning elements are to be arranged between the patient's jaws, the shape and dimensions of the two positioning elements may in each case be selected, for example, such that the shape and dimensions of the two positioning elements in combination with the two drilling templates correspond to the shape and dimensions of the bite register.

By way of example, the pressure which the two jaws apply to the bite register during the recording of the jawbone structure data may also be sensed. This may take place, for example, using a pressure sensing element of the bite register, which senses the grip pressure applied to the bite register. By way of example, the positioning element may also comprise a pressure sensing element, by which the pressure applied to the positioning element by the jaws can be sensed and a check can be carried out to ascertain whether this corresponds to the grip pressure applied to the bite register.

By way of example, by using the positioning element, a drilling template for a jaw can be positioned and fixed, by means of pressure, which the patient applies to the first positioning element by closing the jaw and an opposing jaw, in a position relative to the bone of the jaw which corresponds to the position of the bite register relative to the bone of the jaw during the recording of the jawbone structure data. It can also be ensured that, at the same time, the two jawbones are arranged relative to each other in a position which corresponds to the recording position of the jaws relative to each other during the recording of the jawbone structure data.

By way of example, the positions of implant holes in the jaw or jawbone are planned or defined using the jawbone structure data. The positioning element may have the advantage that it can be used to reproduce the recording situation, i.e. the relative jawbone position and the position of the soft gingiva relative to the associated jawbone during the recording of the jawbone structure data. By using the positioning element to reproduce or simulate the recording situation, it can be ensured, for example, that the situation in which the implant holes are made in the jawbone using the drilling template corresponds to the situation for which the positions of the implant holes were planned or defined. Deviations between the planned positions and the actual positions of the implant holes can thus be minimized and/or eliminated, for example.

The positions of the implant holes are defined, for example, by the drilling template. Since the drilling template can be arranged and fixed in the same position relative to the jaw or jawbone, it can be ensured, for example, that the positions of the implant holes predefined by the drilling template correspond exactly to the planned positions of the implant holes.

Once the drilling template has been positioned and fixed in the intended position relative to a jawbone of the jaw, in a jaw pressure-based manner, by using the positioning element, the implant holes for example can be made in the jawbone at the positions defined by the corresponding through-openings of the drilling template. By way of example, fixing holes can first be made in the jawbone in positions provided for this purpose, into which fixing elements are inserted. These fixing elements are configured, for example, to fix the drilling template, in a jaw pressure-independent manner, in the intended position for making the implant holes. This makes it possible, for example, for the first implant holes to be made in the jaw without the patient applying pressure to the positioning element or the drilling template.

Shape data of a surface of the gingiva of the jaw can be recorded, for example, using an impression of the surface in question, which is recorded by means of a bite register. To this end, the bite register is arranged, for example, between the two jaws of the patient, who exerts pressure on the bite register, using the jaws, in order to create one or two impressions in the bite register. The jaws are located, for example, in the recording position relative to each other, i.e. in a position in which also the jawbone structure data are recorded. The impression of the jaw surface, e.g. gingival surface, defines, for example, the template contact surface of the drilling template or serves as the geometric specification for the latter. Furthermore, while being created, the impression is located in a position relative to the jawbone which is reproduced or simulated by the template contact surface using the positioning element.

By way of example, the shape data may be recorded by a separate scan of the bite register. By way of example, this scan may take place using electromagnetic radiation, for instance in the visible wavelength range. By way of example, this scan may take place using a computed tomography method. Alternatively, this scan may take place using a haptic or tactile scanning method, in which the surfaces of the bite register are scanned with a probe.

By way of example, the shape data may be recorded jointly with the jawbone structure data. By way of example, the material of the bite register used to create the impression is opaque, i.e. visible, for the scanning method used to record the jawbone structure data, and therefore the impression is visible on the scan of the jawbone structure. By way of example, if the impression is not visible, the above-mentioned separate scanning method may be used to record the shape data.

By way of example, structure data of the jawbone may be recorded while the bite register is arranged between the two jaws in the recording position. By way of example, these jawbone structure data may be used to create a digital model of the jaw, which is used to define the implant holes in the jaw. By way of example, the gingiva is transparent, i.e. invisible, for the scanning method used to record the jawbone structure data. Since the implant holes extend, for example, through the gingiva into the jawbone, surface structure data of the gingiva may be necessary in addition to the jawbone structure data in order to define the positions of the implant holes. Said surface structure data of the gingiva may be recorded, for example, using the shape data recorded by means of the bite register. The surface structure data of the gingiva may be used, for example, to create a digital model of the gingiva. In particular, the positions of the mouths of the planned implant holes in the gingival surface may be defined using the surface structure data of the gingiva or the model of the gingiva. These mouths define, for example, the positions of the through-openings of the drilling template resting on the gingival surface. Through these through-openings, the implant holes are made in the jawbone through the gingiva.

By way of example, in order to determine the relative position of the impression comprised by the bite register and thus of the gingival surface relative to the jawbone, which serves as a reference position for positioning the drilling template, position data of the bite register relative to the jawbone are recorded while the bite register is arranged between the two jaws in the recording position.

By way of example, said position data may be recorded jointly with the jawbone structure data. By way of example, the material of the bite register used to create the impression is opaque, i.e. visible, for the scanning method used to record the jawbone structure data. By way of example, the bite register containing the impression is recorded jointly with the jawbone structure data. By way of example, the position data of the bite register relative to the jawbone can be determined from said scan data. By way of example, the material of the bite register used to create the impression may be transparent, i.e. invisible, for the scanning method used to record the jawbone structure data. By way of example, in order that the position of the bite register relative to the jawbone can nevertheless be recorded jointly with the jawbone structure data, the bite register comprises one or more markers which are opaque for the scanning method used to record the jawbone structure data.

By way of example, the recorded position data of the bite register relative to the jawbone are used to position the digital model of the gingiva and/or of the gingival surface relative to the digital model of the jawbone structure. The model of the gingiva or of the gingival surface is based, for example, on the shape data of the impression of the gingival surface in the bite register. The models thus arranged and aligned relative to each other are used, for example, to define the positions of the implant holes through the gingiva into the jawbone.

By way of example, in order to determine the position of the bite register relative to the jawbone, use is made of structure data of a dental object of the jaw which is opaque, i.e. visible, for the scanning method used to record the jawbone structure data and which additionally leaves an impression in the bite register. The dental object can be used as a reference object for determining the relative position, since the position thereof relative to the jawbone is known from the jawbone structure data and the position thereof relative to the bite register is known from the shape data of the impression.

Using the recorded shape data of the impression in the bite register, the jawbone structure data and the position data of the bite register relative to the jawbone, a joint three-dimensional digital model of the jawbone structure and of the gingival surface can be created. This joint model comprises, for example, a three-dimensional digital model of the jawbone structure, which is created using the jawbone structure data, and a three-dimensional digital model of the gingival surface, which is created using the shape data. In the joint model, these two models are arranged, for example, in a position relative to each other which is determined using the position data of the bite register relative to the jawbone.

The joint model is used, for example, to define implant positions for making implant holes in the jaw, i.e. through the gingiva into the jawbone. By way of example, in the course of defining implant positions, three-dimensional digital models of the implant holes are supplemented and positioned in the joint model. By way of example, the joint model is supplemented with models of the implants to be inserted in the implant holes.

By way of example, a three-dimensional digital model of the drilling template and of the positioning element is created using the joint model of jawbone structure and gingival surface. The models of the implant holes, which define the implant positions, can be used, for example, to create through-holes in the model of the drilling template. These through-holes can be used to make the implant holes. By way of example, the mouths of the implant holes in the gingival surface define the positions of the through-openings in the drilling template. By way of example, the positions of the through-openings in the drilling template are selected such that the mouths of the through-openings in the template contact surface coincide with the mouths of the planned implant holes in the gingival surface. By way of example, the model of the drilling template and of the positioning element comprises models of the implants to be inserted.

The shape and dimensions of the model of the positioning element may be determined such that, when pressure is applied to the first positioning element, the two jaws are arranged in the recording position and the drilling template is arranged in a position relative to the jawbone which corresponds to the position of the bite register relative to the jawbone during the recording of the jawbone structure data. By way of example, the shape and dimensions of the positioning element may be determined in dependence on one or two drilling templates and/or a further positioning element such that the two jaws are arranged in the recording position and the drilling template is arranged in the intended position when the positioning element together with the one or two drilling templates and/or the further positioning element is arranged between the two jaws and the two jaws apply pressure to the corresponding templates and elements arranged therebetween.

The model of the positioning element may be used, for instance as a specification, to produce the corresponding positioning element. By way of example, this may have the advantage that, in addition to a CAD method for creating the model of the positioning element, a CAM method for producing the corresponding positioning element can be provided. The positioning element may be printed, for example using a 3D printer, or may be produced by means of a machining device, for instance a material-removing machining device, such as a milling cutter for example.

The positioning element could have the advantage that it enables precise positioning and/or fixing of the drilling template on the jaw. Precise positioning of the drilling template makes it possible to precisely define positions at which planned holes, such as implant holes, are to be made. It can thus be ensured that holes which are made using the drilling template can be made precisely at the intended location.

The positioning element may enable the drilling template to be fixed in a manner true to the recording, i.e. may enable a fixing in which the two jaws are in the recording position relative to each other in which the shape data and structure data were recorded. In this case, it can be ensured that the position and orientation of the template contact surface matches the position and orientation of the impression in the first position relative to the jawbone during the recording of the shape data and structure data.

This could have the advantage that the configuration of the positioning element can be adjusted so as to reproduce the relative jaw position and/or orientation in which the structure data used as the basis for planning the holes were recorded. It can thus be ensured that the execution situation in which the planned holes are executed corresponds exactly to the recording situation or the planning situation, and deviations between planning and execution can be avoided. Consideration can thus primarily be given to the conditions under which the data that serve as a basis for planning the drilling template and the positioning element were recorded. By way of example, questions regarding the future position of the dental prosthesis and/or a resulting occlusion can be taken into account in the course of planning the implant holes, but are not necessarily included in the planning of the positioning element. In other words, the positioning element can be configured to reproduce the recording situation, not to simulate a usage situation with a dental prosthesis anchored by the implants. This could have the advantage that the positioning element does not include a model of the dental prosthesis and does not have to take such a model into account. The planning, production and/or use of the positioning element could thus be simplified.

Furthermore, there could be the advantage that the recording position of the jaws relative to each other can be used directly for planning the drilling template, wherein the positioning element ensures that the relative jaw position during use of the drilling template corresponds to the recording position. By way of example, a single positioning element, for example a positioning element formed in one piece, may be used to arrange and/or to fix the drilling template exactly in the position relative to the jawbone which corresponds to the position during the recording of the shape data and the structure data.

By way of example, a simulation of the recording position is used, for instance in the form of the first digital model, and this is used to determine a relationship between the drilling template and the positioning element that corresponds exactly to the bite register and its positioning during the recording of the shape data and structure data. It can thus be ensured that the shape and dimensions of the positioning element are configured such that, when pressure is applied to the positioning element, the two jaws are arranged in the recording position and the first drilling template is arranged in the first position relative to the jawbone.

This could have the advantage that it is not necessary to take the position of the future dental prosthesis into account when digitizing the bone structure of the jaw and/or when digitizing the support surfaces of the gingiva, i.e. when creating the first digital model.

When defining the implant positions, the structural conditions in and around the jawbone may also be taken into account. It can thus be ensured that these conditions are also taken into account when making the implant holes using the defined implant positions. When defining the implant positions, care can be taken to ensure, for example, that the jaw nerve, i.e. the mandibular nerve, is not damaged when making implant holes at the corresponding implant positions, that the bone wall or bone walls around the implant holes have a sufficient minimum thickness on all sides to be able to provide sufficient support for the implants, that no main blood vessels are hit by the implant holes, and/or that the implant holes each have a correct drilling angle for the implants used. The implant positions for the planned implants can be precisely defined. By way of example, an implant position is to be precisely defined in the submillimeter range, for example to an accuracy of 0.1 mm.

By way of example, 3D scanning and visualization technologies can be used to plan implant holes and to define implant positions. A drilling template can be produced for the planned implant holes, which drilling template makes it possible or ensures that the implant holes are made in the patient's jaw or jawbone exactly at the implant positions planned for the implants to be inserted and/or with the exact depths planned for said implants.

By way of example, a bite register is used to record and determine a jaw relation. Such a register comprises, for example, a plate comprising a wax, plastic and/or a soft metal, which is inserted between the fully toothed, partially toothed or toothless jaws of a patient in order to record or determine the position of the lower jaw relative to the upper jaw. By way of example, a register is used as an intraoral aid for identifying, measuring and fixing jaw positions and jaw contacts of all kinds. By way of example, a bite register is used to determine relative positions of antagonists in the dentition. By way of example, when producing a prosthesis, a bite register can be used to check whether the bite is anatomically optimal. By way of example, a bite register can be used to record a surface, for example a gingival surface, and/or dental objects of one or both jaws.

One dental treatment for a partially or entirely toothless jaw comprises, for example, inserting an anchorage for a prosthesis. By way of example, a dental prosthesis is anchored in the patient's jaw using one or more implants. By way of example, in order to insert the implants, a hole is drilled into the jaw for each of the implants to be inserted, i.e. an implant hole is made which is configured to receive the implant in question. In order to drill such holes, use may be made of drilling templates which define the positions of the holes to be drilled. In order to make the implant holes, the drilling template is arranged on the gingiva of the jaw.

By way of example, the patient's jaw is a toothless jaw. By way of example, the joint model of jawbone structure and gingival surface can be created without requiring teeth or other dental objects as reference objects. By way of example, the positioning element may enable dental treatment of one or two toothless jaws of a patient. By way of example, when pressure is applied to the positioning element by the two jaws, a drilling template can be arranged in an intended position relative to the jawbone. By way of example, this positioning can take place regardless of the presence of teeth or other dental objects in the jaws, i.e. one or both jaws may be toothless. By way of example, the drilling template can be fixed and supported on the toothless jaw using the positioning element, via which the drilling template can be fixed and supported in a jaw pressure-based manner.

By way of example, once a correct position of the drilling template has been achieved, the positioning element can further be used to keep the drilling template constantly in the correct position in a jaw pressure-based manner, without slipping, while the implant holes are being made. By way of example, once a correct position of the drilling template has been achieved, the positioning element can be used to keep the drilling template constantly in the correct position in a jaw pressure-based manner, without slipping, while the fixing holes are being made and the fixing elements are being inserted in the fixing holes. By way of example, the fixing elements can then be used to keep the drilling template constantly in the correct position in a jaw pressure-independent manner, without slipping, while the implant holes are being made. By way of example, following the insertion of the fixing elements, which fix the drilling template on the jaw or jawbone, the patient can stop applying pressure to the positioning element with his jaws. By way of example, the patient may open the jaws in order to make it easier to make the implant holes. By way of example, opening the jaws may facilitate access to the through-openings of the drilling template. By way of example, the positioning element may also be removed from the patient's oral cavity in order to make it even easier to make the implant holes. By way of example, removing the positioning element may further facilitate access to the through-openings of the drilling template.

In the case of a toothless, i.e. edentulous, jaw, the use of a drilling plate presents several challenges. In the case of a toothless jaw, apart from the jawbone, which serves as an internal reference structure, there are no external reference structures on the jaw that could be used to position the drilling template exactly on the jaw relative to the jawbone.

The positioning of a drilling template may thus prove to be a challenge. Besides the positioning, it may also prove to be difficult to provide sufficient support for the drilling template in the case of a toothless jaw. A toothless jaw lacks structures, such as teeth, on which such a drilling template can be fixed and/or supported. Rather, in this case, a drilling template sits on the soft, yielding gingiva, i.e. the gums, of the jaw. The drilling template thus lacks support since it can move on and/or with the gingiva. Using the method described herein, it is possible to provide, for example, a positioning element which enables a drilling template to be both positioned and fixed on a toothless jaw. Positioning and fixing using the positioning element takes place, for example, in a jaw pressure-based or jaw pressure-dependent manner. A positioning between the jaws may take place, which is fixed by the pressure. The opposing jaws with their complex surface structures can uniquely define the position of the combination of drilling template(s) and positioning element(s) arranged therebetween. For example, for a given jaw, the opposing jaw can serve as a reference structure.

Even once a correct position has been achieved, it can generally be difficult to keep the drilling template constantly in the correct position, without slipping, while implant holes are being made. By way of example, the positioning element can make it possible to keep the drilling template constantly in the correct position in a jaw pressure-based manner, without slipping, while the implant holes are being made.

By way of example, the first positioning element is configured to fix the first drilling template in the first position for making first fixing holes in the jaw for first fixing elements. By way of example, the first drilling template comprises second through-openings. The second through-openings each define a first fixing position for making one of the first fixing holes. By way of example, the first fixing elements are configured to fix the first drilling template, in a jaw pressure-independent manner, in the first position for making the first implant holes in the jaw for the first dental implants. By way of example, the method further comprises defining the first fixing positions for making the first fixing holes in the jaw using the first model. By way of example, the second three-dimensional digital model of the first drilling template and of the first positioning element is also created using defined first fixing positions.

This could have the advantage of making it possible for the drilling template to be fixed, in a jaw pressure-independent manner, in the intended position for making the implant holes in the jaw. The jaw pressure-independent fixing may in particular take place without using the positioning element. By way of example, the positioning element may be removed from the patient's oral cavity following insertion of the fixing elements. By way of example, the drilling template for making the fixing holes in the jaw is fixed in the intended position, in a jaw pressure-dependent or jaw pressure-based manner, using the positioning element.

By way of example the planning of the fixing holes may take place in a manner analogous to the planning of the implant holes. By way of example, positions of the fixing holes are defined using the joint model of the jawbones and of the gingival surface. By way of example, the joint model is supplemented with three-dimensional digital models of the fixing holes. By way of example, the joint model is supplemented with three-dimensional digital models of the fixing elements to be inserted in the fixing holes.

By way of example, the model of the drilling template in the model of the drilling template and of the positioning element is configured such that it comprises additional through-openings for making the fixing holes in the jawbone, through the gingiva, at the positions intended in the joint model. By way of example, the positions of the additional through-openings of the drilling template are selected such that the mouths of the additional through-openings in the template contact surface coincide with the mouths of the planned fixing holes in the gingival surface. By way of example, the model of the drilling template and of the positioning element is supplemented with three-dimensional digital models of the fixing elements to be inserted in the fixing holes.

By way of example, in order to keep the drilling template for the implant holes constantly in a fixed position relative to the jaw, use could be made of fixing elements. Besides implant holes, the drilling template could also define additional fixing holes for inserting fixing elements into the jaw. The fixing elements may be used to fix the drilling template in a stable position on the jaw, in which case the fixing elements provide support for the drilling template on the soft gingiva. In the case of a toothless jaw, the same difficulties as when making implant holes may generally arise when making the fixing holes: difficulties may arise both in positioning the drilling template and in keeping the drilling template constantly in an appropriate position for making the fixing holes. The drilling template may lack position references and support on the soft gingiva, and therefore there is a risk of the drilling template slipping while being positioned and/or while the fixing holes are being made. However, if the fixing holes deviate from their planned positions in the jaw, the position of the drilling template and thus also the positions predefined by the drilling template for the implant holes consequently deviate from the planned positions. By way of example, these difficulties could be avoided by using a positioning element for the drilling template. The positioning element can enable the drilling template to be positioned and fixed at an intended position relative to the jawbone. The through-openings for making the fixing holes in the jaw can thus be positioned at the intended positions relative to the gingiva and the jawbone. While the fixing holes are being made in the jaw and the fixing elements are being inserted in the fixing holes, the drilling template can be kept in the intended position relative to the jawbone, in a jaw pressure-based manner, by using the positioning element. The inserted fixing elements can thus also fix the drilling template in the intended position. This fixing using the fixing element is independent of the jaw pressure, i.e. it enables the jaws to be opened and/or the positioning element to be removed without altering the position of the drilling template relative to the jawbone.

This could have the advantage that the positioning element enables precise positioning and fixing of the drilling template on the jaw even before the fixing holes are made. It can thus be ensured that the fixing holes and thus the implant holes to be made subsequently also take place precisely at the intended positions.

If a drilling template lacks support, for example in the case of a toothless jaw, the drilling template may move on the soft gingiva, making the position information provided by the drilling template inaccurate and thus incorrect. By way of example, in order to keep the drilling template for the implant holes firmly in the first position, use is made of fixing elements which are inserted into the jaw laterally, for instance labially and/or buccally. To this end, the drilling template defines, for example, fixing positions which are positions at which fixing holes for receiving corresponding fixing elements are made. By way of example, these fixing holes are also planned in the drilling template in addition to the implant holes, and therefore the drilling template also includes these.

The same problem as for the implant holes also arises for fixing holes in the case of a toothless jaw. Since the gingiva is soft and lacks both reference structures for positioning purposes and fixing structures for fixing the drilling template on the jaw once positioned, there is a risk that the drilling template will also slip while the fixing holes are being made. This risk can be avoided by using of a positioning element.

Furthermore, there could be the advantage that it is not necessary to take into account the position of the future dental prosthesis during the drilling and the insertion of the fixing elements. Since the precise conditions under which the shape data and structure data were recorded are exactly reproduced when the drilling template is fixed in a pressure-based manner using the positioning element, a highly precise positioning of the fixing holes and thus of the implant holes can be achieved, which corresponds exactly to the planning specifications. Fixing the drilling template in a jaw pressure-based manner refers to an application of pressure to the positioning element by the jaws, wherein the jaws are arranged in the recording position and the drilling template is arranged in the predetermined position relative to the jawbone. To this end, for example, a suitable shape and suitable dimensions of the positioning element are determined using the second digital model.

By way of example, the method further comprises producing the first drilling template, using the second three-dimensional digital model. This could have the advantage that, in addition to the positioning element, the drilling template for which the positioning element is configured can also be provided.

The model of the drilling template may be used, for instance as a specification, to produce the corresponding drilling template. By way of example, this may have the advantage that, in addition to a CAD method for creating the model of the drilling template, a CAM method for producing the corresponding drilling template can also be provided. The drilling template may be printed, for example using a 3D printer, or may be produced by means of a machining device, for instance a material-removing machining device, such as a milling cutter for example.

By way of example, the first positioning element comprises one or more first connecting elements, which are configured to establish and to release a non-destructively releasable first connection between the first positioning element and the first drilling template in the patient's oral cavity.

This could have the advantage that the positioning element can be removed from the patient's oral cavity, while the drilling template remains in the oral cavity. By way of example, the drilling template remaining in the oral cavity may be fixed to the jaw using fixing elements. By way of example, when inserting the drilling template and the positioning element into the patient's oral cavity, the drilling template and the positioning element may be connected via the non-destructively releasable connection and may be inserted jointly. By way of example, the drilling template and the positioning element may be inserted into the patient's oral cavity one after the other and may be connected to each other therein using the non-destructively releasable connection.

By way of example, the non-destructively releasable first connection comprises a plug-in connection. This could have the advantage that an efficient, non-destructively releasable connection can be provided. By way of example, the positioning element and/or the drilling template comprise one or more female connecting elements, which are configured to receive one or more male connecting elements of the drilling template and/or of the positioning element.

By way of example, the first drilling template comprises the first positioning element. By way of example, the first positioning element comprises first access openings to the first through-openings of the first drilling template. This could have the advantage that the positioning element can remain in the patient's oral cavity in order to make the implant holes. Access to the through-openings of the drilling template to make the implant holes is provided, for example, via the access openings of the positioning element.

By way of example, the first access openings are accessible to make the implant holes while pressure is being exerted on the positioning element by the jaws. By way of example, the first access openings are accessible to make the implant holes when no pressure is being exerted on the positioning element by the jaws and the jaws are spaced further apart than is the case in the recording position. By way of example, the jaws must be spaced so far apart that the opposing jaw or a second drilling template loses contact with the positioning element. By way of example, the first access openings are designed as channels, each leading to one of the through-openings. By way of example, the first access openings are designed as an aperture in the positioning element. By way of example, the positioning element has at least in part a lattice structure, the interstices of which provide the apertures. By way of example, the positioning element is open in the vestibular direction, for example labially and/or buccally. By way of example, the positioning element is closed palatinally and/or lingually. By way of example, the positioning element is hollowed out. By way of example, in order to support the cavity, the positioning element comprises one or more support struts which extend for example vertically between the two jaws.

By way of example, second shape data of a second impression of a surface of the opposing jaw are recorded, using the bite register arranged between the two jaws in the recording position, said second shape data being used to create the first three-dimensional digital model, which further comprises the second surface of the opposing jaw. By way of example, the second surface of the opposing jaw may be used as a specification for the geometric shape of a jaw contact surface of the positioning element or a template contact surface of a second drilling template.

By way of example, the first positioning element comprises a jaw contact surface for making contact with the surface of the opposing jaw. The second impression defines the jaw contact surface. Via the jaw contact surface, the opposing jaw can exert pressure for example directly on the positioning element.

By way of example, the method further comprises recording second structure data of an opposing jawbone of the opposing jaw while the bite register is arranged between the two jaws in the recording position. By way of example, the opposing jawbone structure data are recorded jointly with the jawbone structure data. The second structure data are used to create the first three-dimensional digital model, which further comprises the opposing jawbone structure. The method further comprises producing a second drilling template, which comprises a second template contact surface for making contact with the surface of the opposing jaw. The second impression defines the second template contact surface. The surface of the opposing jaw is a surface of the gingiva of the opposing jaw. The second drilling template further comprises third through-openings. The third through-openings each define a second implant position for making a second implant hole in the opposing jaw for second dental implants. The recorded position data comprise position data of the bite register relative to the opposing jawbone. The method further comprises defining the second implant positions for inserting the second implants in the opposing jaw, using the first model. The second model comprises the second drilling template. The defined second implant positions are also used to create the second model.

This could have the advantage that, in addition to a drilling template for making implant holes in the jaw, a drilling template for making implant holes in the opposing jaw can also be created. Using the positioning element, for example, the two drilling templates can each be positioned and fixed, in a pressure-based manner, in a position relative to the jawbone or the opposing jawbone which corresponds to a position of the bite register relative to the jaw or the opposing jaw during the recording of the jawbone structure data or the opposing jawbone structure data.

By way of example, the patient's opposing jaw is a toothless opposing jaw.

By way of example, the first positioning element is further configured to fix the second drilling template in the second position by means of pressure, which the patient applies to the first positioning element by closing the two jaws. The shape and dimensions of the first positioning element are further determined such that the second drilling template is arranged in the second position relative to the opposing jaw.

This could have the advantage that the second drilling template or the second template contact surface can be positioned and fixed in a second position relative to the opposing jaw which corresponds to the position of the bite register or of the second impression relative to the opposing jawbone during the recording of the structure data of the opposing jawbone or simulates the corresponding reference position.

By way of example, the first positioning element is configured to fix the second drilling template in the second position for making second fixing holes in the opposing jaw for second fixing elements. By way of example, the second drilling template further comprises fourth through-openings which each define a second fixing position for making one of the second fixing holes. By way of example, the second fixing elements are configured to fix the second drilling template, in a jaw pressure-independent manner, in a second position relative to the opposing jawbone for making second implant holes in the opposing jaw for second dental implants. By way of example, the method further comprises defining the second fixing positions for inserting the second fixing elements in the opposing jaw, using the first model. By way of example, the defined second fixing positions are also used to create the second model.

This could have the advantage that the second drilling template can be positioned and configured in the second position in a jaw pressure-dependent manner. This jaw pressure-dependent positioning and fixing can be used to make fixing holes and to insert fixing elements in the opposing jaw.

This could have the advantage that a jaw pressure-independent fixing of the drilling template in the second position for making the implant holes in the opposing jaw can be made possible by means of fixing elements. The jaw pressure-independent fixing may in particular take place without using the positioning element. By way of example, the positioning element may be removed from the patient's oral cavity following the insertion of the fixing elements.

By way of example, the planning of the second fixing holes may take place in a manner analogous to the planning of the first fixing holes. By way of example, positions of the fixing holes in the opposing jaw are defined using the joint model of the jawbones and of the gingival surfaces. By way of example, the joint model is supplemented with three-dimensional digital models of the fixing holes in the opposing jaw. By way of example, the joint model is supplemented with three-dimensional digital models of the fixing elements to be inserted in the fixing holes in the opposing jaw.

By way of example, the model of the second drilling template in the model of the drilling templates and of the positioning element is configured such that the second drilling template comprises additional through-openings for making the fixing holes in the opposing jawbone, through the gingiva, at the positions intended in the joint model. By way of example, the positions of the additional through-openings of the second drilling template are selected such that the mouths of the additional through-openings in the template contact surface coincide with the mouths of the planned fixing holes in the gingival surface of the opposing jaw. By way of example, the model of the drilling templates and of the positioning element is supplemented with three-dimensional digital models of the fixing elements to be inserted in the fixing holes in the opposing jaw.

By way of example, the first positioning element is further configured to fix the second drilling template in the second position for making the second fixing holes in the opposing jaw for the second fixing elements.

By way of example, the first positioning element further comprises one or more second connecting elements, which are configured to establish and to release a non-destructively releasable second connection between the first positioning element and the second drilling template in the patient's oral cavity.

This could have the advantage that the positioning element can be removed from the patient's oral cavity while the second drilling template together with the first drilling template remains in the oral cavity. By way of example, the second drilling template remaining in the oral cavity may be fixed to the opposing jaw using fixing elements. By way of example, when inserting the two drilling templates and the positioning element into the patient's oral cavity, one or two of the two drilling templates and the positioning element may be connected via the relevant non-destructively releasable connection and may be inserted jointly. By way of example, the drilling templates and the positioning element may be inserted into the patient's oral cavity one after the other and may be connected to each other therein using the corresponding non-destructively releasable connections.

By way of example, the non-destructively releasable second connection comprises a plug-in connection. This could have the advantage that an efficient, non-destructively releasable connection can be provided. By way of example, the positioning element and/or the second drilling template comprise one or more female connecting elements, which are configured to receive one or more male connecting elements of the second drilling template and/or of the positioning element.

By way of example, the second drilling template comprises the first positioning element.

By way of example, the first positioning element comprises second access openings to the third through-openings of the second drilling template. This could have the advantage that the positioning element can remain in the patient's oral cavity in order to make the implant holes in the opposing jaw. Access to the through-openings of the second drilling template to make the implant holes in the opposing jaw is provided, for example, via the access openings of the positioning element.

By way of example, the second access openings are accessible to make the implant holes in the opposing jaw while pressure is being exerted on the positioning element by the jaws. By way of example, the second access openings are accessible to make the implant holes in the opposing jaw when no pressure is being exerted on the positioning element by the jaws and the jaws are spaced further apart than is the case in the recording position. By way of example, the jaws must be spaced so far apart that the first drilling template loses contact with the positioning element. By way of example, the second access openings are designed as channels, each leading to one of the through-openings. By way of example, the second access openings are designed as an aperture in the positioning element. By way of example, the positioning element has at least in part a lattice structure, the interstices of which provide the apertures. By way of example, the positioning element is open in the vestibular direction, for example labially and/or buccally. By way of example, the positioning element is closed palatinally and/or lingually. By way of example, the positioning element is hollowed out. By way of example, in order to support the cavity, the positioning element comprises one or more support struts which extend for example vertically between the two jaws.

By way of example, the first and second drilling template jointly comprise the first positioning element. This could have the advantage that a common element can be provided, which comprises the two drilling templates and the first positioning element. This common element can be inserted in the patient's oral cavity. When the patient uses the jaws to apply pressure to this common element, more precisely to the two drilling templates and thus to the positioning element, the two drilling templates can each be positioned and fixed for example in an intended position relative to the jaw and to the opposing jaw, respectively. By way of example, the two jaws can thus also be arranged relative to each other in the recording position.

By way of example, the method further comprises producing a second positioning element. By way of example, the production of the second drilling template further comprises producing the second positioning element. By way of example, the second positioning element in combination with the first positioning element is configured to fix the second drilling template in the second position by means of pressure, which the patient applies to the first and second positioning element by closing the jaws. The shape and dimensions of the second positioning element are determined such that, when the pressure is applied to the first and second positioning element, the two jaws are arranged in the recording position and the second drilling template is arranged in the second position relative to the opposing jaw.

This could have the advantage that a positioning element can be provided for each of the drilling templates. The two positioning elements in combination with each other can respectively position and fix the two drilling templates in the intended position relative to the jawbone and the opposing jawbone.

By way of example, the second positioning element in combination with the first positioning element is configured to fix the second drilling template in the second position for making the second fixing holes in the opposing jaw for the second fixing elements.

By way of example, the second positioning element comprises one or more third connecting elements, which are configured to establish and to release a non-destructively releasable third connection between the second positioning element and the second drilling template in the patient's oral cavity.

This could have the advantage that the second positioning element can be removed from the patient's oral cavity while the second drilling template together with the first drilling template remains in the oral cavity. By way of example, the second drilling template remaining in the oral cavity may be fixed to the opposing jaw using fixing elements. By way of example, when inserting the two drilling templates and the two positioning elements into the patient's oral cavity, the drilling templates and the positioning elements may be connected in pairs via corresponding non-destructively releasable connections and may be inserted jointly. By way of example, the drilling templates and positioning elements may be inserted into the patient's oral cavity one after the other and may be connected to each other therein using the corresponding non-destructively releasable connections.

By way of example, the non-destructively releasable third connection comprises a plug-in connection. This could have the advantage that an efficient, non-destructively releasable connection can be provided. By way of example, the second positioning element and/or the second drilling template comprise one or more female connecting elements, which are configured to receive one or more male connecting elements of the second drilling template and/or of the second positioning element.

By way of example, the second positioning element is comprised by the second drilling template. By way of example, the second positioning element comprises second access openings to the third through-openings of the second drilling template. This could have the advantage that the second positioning element can remain in the patient's oral cavity in order to make the implant holes in the opposing jaw. Access to the through-openings of the second drilling template to make the implant holes in the opposing jaw is provided, for example, via the access openings of the second positioning element.

By way of example, the second access openings are accessible to make the implant holes in the opposing jaw while pressure is being exerted on the positioning elements by the jaws. By way of example, the second access openings are accessible to make the implant holes in the opposing jaw when no pressure is being exerted on the positioning elements by the jaws and the jaws are spaced further apart than is the case in the recording position. By way of example, the jaws must be spaced so far apart that the first positioning element loses contact with the second positioning element. By way of example, the second access openings are designed as channels, each leading to one of the through-openings. By way of example, the second access openings are designed as an aperture in the second positioning element. By way of example, the second positioning element has at least in part a lattice structure, the interstices of which provide the apertures. By way of example, the second positioning element is open in the vestibular direction, for example labially and/or buccally.

By way of example, the second positioning element is closed palatinally and/or lingually. By way of example, the second positioning element is hollowed out. By way of example, in order to support the cavity, the second positioning element comprises one or more support struts which extend for example vertically between the two jaws.

By way of example, the first positioning element comprises one or more fourth connecting elements and the second positioning element comprises one or more fifth connecting elements. The fourth and fifth connecting elements are configured to establish and to release a non-destructively releasable fourth connection between the two positioning elements in the patient's oral cavity.

This could have the advantage that the connection between the two positioning elements in the patient's oral cavity can be released. This may take place, for example, in order to make it easier to make the implant holes in the jaws while the positioning elements remain in the patient's oral cavity. This could have the advantage that the two positioning elements can be removed from the patient's oral cavity independently of each other, for example one after the other, while the drilling templates remain in the oral cavity. By way of example, just one of the two positioning elements is removed from the oral cavity. By way of example, both positioning elements are removed from the oral cavity. By way of example, the drilling templates remaining in the oral cavity may be fixed to the jaws using fixing elements. By way of example, when inserting the drilling templates and positioning elements into the patient's oral cavity, the drilling templates and positioning elements may be connected in pairs via the corresponding non-destructively releasable connections and may be inserted jointly. By way of example, the two drilling templates and the two positioning elements may be jointly inserted into the oral cavity in a manner releasably connected to each other. By way of example, the drilling templates and positioning elements may be inserted into the patient's oral cavity one after the other and may be gradually connected to each other therein using the corresponding non-destructively releasable connections.

By way of example, the non-destructively releasable fourth connection comprises a plug-in connection. This could have the advantage that an efficient, non-destructively releasable connection can be provided. By way of example, the first positioning element and/or the second positioning element comprise one or more female connecting elements, which are configured to receive one or more male connecting elements of the second positioning element and/or of the first positioning element.

By way of example, the second implant positions of the second implant holes and/or second fixing positions of the second fixing holes are each defined in such a way as to satisfy one or more of the following first positioning criteria: extending the second implant holes and/or second fixing holes through a bone wall of the opposing jawbone structure having a predefined first minimum thickness, maintaining a predefined first minimum distance of the second implant holes and/or second fixing holes from main nerves in the opposing jaw, maintaining a predefined second minimum distance of the second implant holes and/or second fixing holes from blood vessels in the opposing jaw.

By way of example, this could have the advantage that sufficient support for the implants and/or fixing elements can be ensured by maintaining the minimum thickness of the bone wall. Injury to main nerves can be prevented by maintaining the minimum distance from the main nerves in question. Injury to blood vessels can be prevented by maintaining the minimum distance from the blood vessels in question.

By way of example, the second drilling template for the third through-openings and/or fourth through-openings is provided in each case with a drilling guide element, which is configured to guide a drilling tool when making the second implant holes and/or second fixing holes and/or to limit a first drilling depth of the second implant holes and/or second fixing holes.

This could have the advantage that the drilling tool can be guided by the drilling guide elements in each case. By way of example, the drilling guide elements may be provided by the third through-openings and/or fourth through-openings, which are configured at least in part as channels which guide the drilling tool. By way of example, the drilling guide elements are each arranged at the third through-openings and/or fourth through-openings, for example in the form of extensions which each have a through-channel for guiding the drilling tool. By way of example, the drilling guide elements each comprise a stop, by which it is possible to limit a deeper insertion of the drilling tool into the drilling guide element and thus the drilling depth. By way of example, the stop may be provided by a mouth of a channel of the drilling guide element or by a transition between two diameters of the channel of the drilling guide element. By way of example, an inner diameter is smaller than an outer diameter. By way of example, a portion of a holding element of the drilling tool or a drill bit strikes against the stop.

By way of example, the first and/or the second positioning element for the third through-openings and/or fourth through-openings are provided in each case with a third drilling guide element, which is configured to guide the drilling tool when making the second implant holes and/or second fixing holes and/or to limit the first drilling depth of the second implant holes and/or second fixing holes.

This could have the advantage that the drilling tool can be guided by the drilling guide elements in each case. By way of example, the drilling guide elements for the third through-openings may be provided by the second access openings, which are configured at least in part as channels which guide the drilling tool. By way of example, the drilling guide elements are each arranged at the third through-openings and/or fourth through-openings, for example in the form of extensions which each have a through-channel for guiding the drilling tool. By way of example, the drilling guide elements each comprise a stop, by which it is possible to limit a deeper insertion of the drilling tool into the drilling guide element and thus the drilling depth. By way of example, the stop may be provided by a mouth of a channel of the drilling guide element or by a transition between two diameters of the channel of the drilling guide element. By way of example, an inner diameter is smaller than an outer diameter. By way of example, a portion of a holding element of the drilling tool or a drill bit strikes against the stop.

By way of example, the first implant positions of the first implant holes and/or first fixing positions of the first fixing holes are each defined in such a way as to satisfy one or more of the following second positioning criteria: extending the first implant holes and/or first fixing holes through a bone wall of the jawbone structure having a predefined second minimum thickness, maintaining a predefined third minimum distance of the first implant holes and/or first fixing holes from main nerves in the jaw, maintaining a predefined fourth minimum distance of the first implant holes and/or first fixing holes from blood vessels in the jaw.

By way of example, this could have the advantage that sufficient support for the implants and/or fixing elements can be ensured by maintaining the minimum thickness of the bone wall. Injury to main nerves can be prevented by maintaining the minimum distance from the main nerves in question. Injury to blood vessels can be prevented by maintaining the minimum distance from the blood vessels in question.

By way of example, the first drilling template for the first through-openings and/or second through-openings is provided in each case with a third drilling guide element, which is configured to guide the drilling tool when making the first implant holes and/or first fixing holes and/or to limit a second drilling depth of the first implant holes and/or first fixing holes.

This could have the advantage that the drilling tool can be guided by the drilling guide elements in each case. By way of example, the drilling guide elements may be provided by the first through-openings and/or second through-openings, which are configured at least in part as channels which guide the drilling tool. By way of example, the drilling guide elements are each arranged at the first through-openings and/or second through-openings, for example in the form of extensions which each have a through-channel for guiding the drilling tool. By way of example, the drilling guide elements each comprise a stop, by which it is possible to limit a deeper insertion of the drilling tool into the drilling guide element and thus the drilling depth. By way of example, the stop may be provided by a mouth of a channel of the drilling guide element or by a transition between two diameters of the channel of the drilling guide element. By way of example, an inner diameter is smaller than an outer diameter. By way of example, a portion of a holding element of the drilling tool or a drill bit strikes against the stop.

By way of example, the first positioning element for the first through-openings and/or second through-openings is provided in each case with a fourth drilling guide element, which is configured to guide the drilling tool when making the first implant holes and/or first fixing holes and/or to limit the second drilling depth of the first implant holes and/or first fixing holes.

This could have the advantage that the drilling tool can be guided by the drilling guide elements in each case. By way of example, the drilling guide elements for the first through-openings may be provided by the first access openings, which are configured at least in part as channels which guide the drilling tool. By way of example, the drilling guide elements are each arranged at the first through-openings and/or second through-openings, for example in the form of extensions which each have a through-channel for guiding the drilling tool. By way of example, the drilling guide elements each comprise a stop, by which it is possible to limit a deeper insertion of the drilling tool into the drilling guide element and thus the drilling depth. By way of example, the stop may be provided by a mouth of a channel of the drilling guide element or by a transition between two diameters of the channel of the drilling guide element. By way of example, an inner diameter is smaller than an outer diameter. By way of example, a portion of a holding element of the drilling tool or a drill bit strikes against the stop.

By way of example, the first and/or second structure data are recorded using a first scanning method, which is one of the following methods: a computed tomography method, a volumetric tomography method or a magnetic resonance imaging method. This could have the advantage that, for recording the first and/or second structure data, use can be made for example of a scanning method which is optimized for recording the structure data in question.

By way of example, the position data of the bite register are recorded using material which is opaque for the first scanning method. This could have the advantage that the bite register is at least partially visible for the first scanning method.

By way of example, the position data of the bite register are recorded using markers, which are comprised by the bite register and are opaque for the first scanning method. This could have the advantage that at least the markers are visible for the first scanning method, and the position data of the bite register relative to the jaw and/or opposing jaw can be determined on the basis of the position data of the markers relative to the jaw and/or opposing jaw. By way of example, the bite register comprises at least three markers, which are not arranged on a common straight line. By way of example, the bite register comprises more than three markers.

By way of example, the position data of the bite register are recorded using a contact and/or using contact points between the second impression and a dental object of the opposing jaw which is opaque for the first scanning procedure. This could have the advantage that the position data of the bite register relative to the jawbone and/or opposing jawbone can be determined on the basis of the position of the dental object, or of a portion of the contact of the dental object that comes into contact with the second impression, relative to the jawbone.

By way of example, the first and/or second shape data are recorded using a second scanning method, which is one of the following methods: an optical method, a computed tomography method, a haptic or tactile method. This could have the advantage that, for recording the first and/or second shape data, use can be made for example of a scanning method which is optimized for recording the shape data in question.

By way of example, the markers of the bite register which are opaque, i.e. visible, for the first scanning method are also opaque, i.e. visible, for the second scanning method.

By way of example, the method further comprises sensing a grip pressure applied to the bite register arranged between the patient's two jaws. By way of example, the first positioning element and/or the second positioning element comprises a pressure sensing element for checking whether the pressure applied to the first positioning element and/or the second positioning element in the recording position of the two jaws matches the grip pressure.

This could have the advantage that it can be ensured that, when positioning and/or fixing the drilling template or drilling templates, the same pressure is applied to the positioning element or positioning elements by the jaws as when recording the structure data, i.e. the grip pressure. The pressure applied by the jaws can lead to deformations of the soft gingiva. Using the same pressure may have the advantage of causing the same deformations of the gingiva. By way of example, the bite register also comprises a pressure sensing element.

By way of example, the pressure sensing element may comprise a piezoelectric sensor, a pressure-dependent shape element and/or a pressure-dependent color element. By way of example, the shape of the pressure-dependent shape elements changes as a function of the applied pressure, or the shape of the pressure-dependent shape elements becomes increasingly pronounced, for example, as the pressure increases. By way of example, the color of the pressure-dependent color elements changes as a function of the pressure applied. By way of example, a pressure-sensitive ink is used.

By way of example, a positioning element for fixing a first drilling template on a gingiva of a patient's jaw in a first position relative to a jawbone of the jaw is also provided. The positioning element is configured to fix the first drilling template, by means of pressure, which the patient applies to the positioning element by closing the jaw and an opposing jaw, in the first position for making first implant holes in the jaw for first dental implants. The positioning element is produced using a method which comprises:
recording first shape data of a first impression of the surface of the gingiva of the jaw, using a bite register arranged between the patient's two jaws, wherein the two jaws are located in a recording position relative to each other, wherein the first impression is located in the first position relative to the jawbone, and wherein the first impression defines a first template contact surface of a first drilling template for making contact with a surface of the gingiva of the jaw,
recording first structure data of the jawbone while the bite register is arranged between the two jaws in the recording position,
recording position data of the bite register relative to the jawbone while the bite register is arranged between the two jaws in the recording position,
creating a first three-dimensional digital model, using the recorded first shape data, first structure data and position data, wherein the first model comprises the jawbone structure and the surface of the gingiva of the jaw,
defining first implant positions for making the first implant holes in the jaw, using the first model,
creating a second three-dimensional digital model of the first drilling template and of the positioning element, using the first model and the defined first implant positions, wherein the shape and dimensions of the positioning element are determined such that, when pressure is applied to the positioning element, the two jaws are arranged in the recording position and the first drilling template is arranged in the first position relative to the jawbone, wherein the first drilling template comprises the first template contact surface and first through-openings, wherein the first through-openings each define one of the first implant positions,
wherein the second three-dimensional digital model is used to produce the positioning element.

By way of example, the positioning element is a positioning element produced by one of the above-described methods for producing a positioning element. By way of example, the patient's jaw is a toothless jaw.

By way of example, the positioning element is configured to fix the first drilling template in the first position for making first fixing holes in the jaw for first fixing elements. By way of example, the first drilling template comprises second through-openings which each define one of the first fixing positions. By way of example, the first fixing elements are configured to fix the first drilling template, in a jaw pressure-independent manner, in the first position for making the first implant holes in the jaw for the first dental implants. By way of example, the method further comprises defining first fixing positions for making the first fixing holes and first implant positions for making the first implant holes in the jaw, using the first model. By way of example, the second three-dimensional digital model of the first drilling template and of the positioning element is also created using the defined first fixing positions.

By way of example, a combination of the positioning element according to any one of the preceding examples of positioning elements and a first drilling template produced using the second three-dimensional digital model is also provided.

By way of example, the first drilling template is a drilling template produced by one of the above-described methods for producing a drilling template.

By way of example, the positioning element comprises one or more first connecting elements, which are configured to establish and to release a non-destructively releasable first connection between the positioning element and the first drilling template in the patient's oral cavity.

By way of example, the first drilling template comprises the positioning element. By way of example, the positioning element comprises first access openings to the first through-openings of the first drilling template.

By way of example, the positioning element comprises a jaw contact surface for making contact with a surface of the opposing jaw. The jaw contact surface is defined by a second impression of the surface of the opposing jaw, which is recorded using the bite register arranged between the two jaws in the recording position.

By way of example, the combination further comprises a second drilling template which comprises a second template contact surface for making contact with the surface of the opposing jaw. The second impression defines the second template contact surface. The surface of the opposing jaw is a surface of the gingiva of the opposing jaw. The second drilling template further comprises third through-openings. The third through-openings each define a second implant position for making one of the second implant holes.

By way of example, the second drilling template is a drilling template produced by one of the above-described methods for producing a drilling template.

By way of example, the positioning element is further configured to fix the second drilling template in the second position by means of pressure, which the patient applies to the positioning element by closing the two jaws. The shape and dimensions of the positioning element are further determined such that the second drilling template is arranged in the second position relative to the opposing jaw.

By way of example, the positioning element is configured to fix the second drilling template in the second position for making second fixing holes in the opposing jaw for second fixing elements. By way of example, the second drilling template further comprises fourth through-openings, which each define a second fixing position for making one of the second fixing holes. By way of example, the second fixing elements are configured to fix the second drilling template, in a jaw pressure-independent manner, in a second position relative to the opposing jawbone for making second implant holes in the opposing jaw for second dental implants.

By way of example, the positioning element is further configured to fix the second drilling template in the second position for making the second fixing holes in the opposing jaw for the second fixing elements.

By way of example, the positioning element further comprises one or more second connecting elements, which are configured to establish and to release a non-destructively releasable second connection between the positioning element and the second drilling template in the patient's oral cavity.

By way of example, the second drilling template comprises the positioning element. By way of example, the positioning element comprises second access openings to the third through-openings of the second drilling template. By way of example, the first and second drilling template jointly comprise the positioning element.

By way of example, the combination further comprises a second positioning element. The second positioning element in combination with the positioning element is configured to fix the second drilling template in the second position by means of pressure, which the patient applies to the two positioning elements by closing the jaws. The shape and dimensions of the second positioning element are determined such that, when the pressure is applied to the two positioning elements, the two jaws are arranged in the recording position and the second drilling template is arranged in the second position relative to the opposing jaw.

By way of example, the second positioning element is a positioning element produced by one of the above-described methods for producing a positioning element.

By way of example, the second positioning element in combination with the positioning element is configured to fix the second drilling template in the second position for making the second fixing holes in the opposing jaw for the second fixing elements.

By way of example, the second positioning element comprises one or more third connecting elements, which are configured to establish and to release a non-destructively releasable third connection between the second positioning element and the second drilling template in the patient's oral cavity.

By way of example, the second positioning element is comprised by the second drilling template. By way of example, the second positioning element comprises second access openings to the third through-openings of the second drilling template.

By way of example, the positioning element comprises one or more fourth connecting elements and the second positioning element comprises one or more fifth connecting elements. The fourth and fifth connecting elements are configured to establish and to release a non-destructively releasable fourth connection between the first and the second positioning element in the patient's oral cavity.

Examples of the invention will be explained in greater detail below with reference to the drawings, in which:

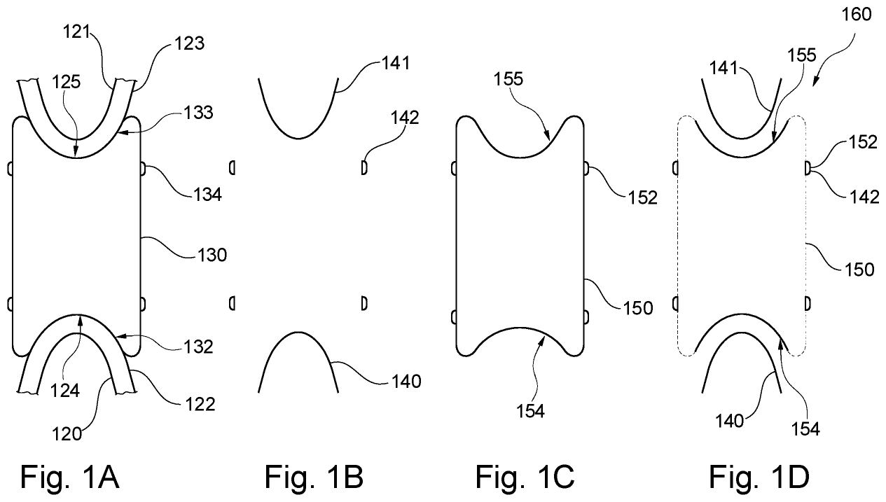

FIG. 1 show an exemplary bite register between jaws, as well as data of the jawbones and of the bite register,

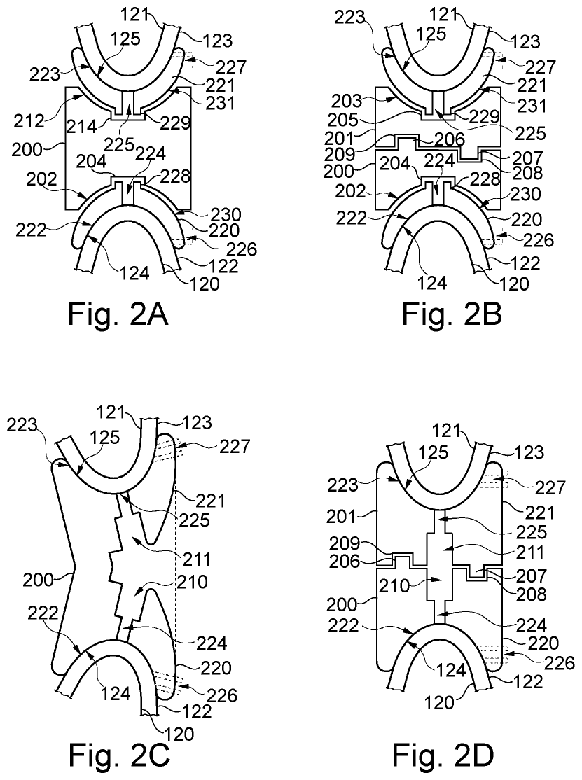

FIG. 2 show exemplary positioning elements and drilling templates,

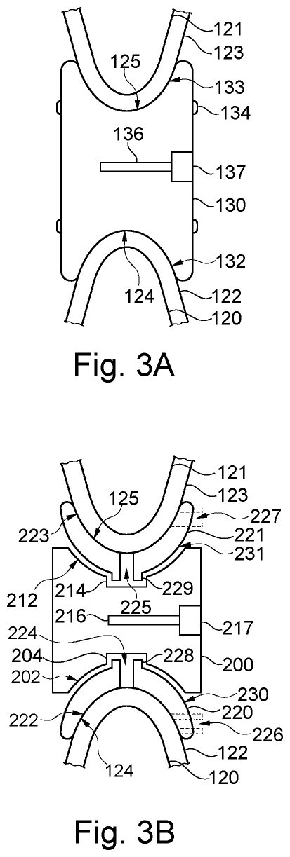

FIG. 3 show an exemplary bite register and an exemplary positioning element with pressure sensing elements,

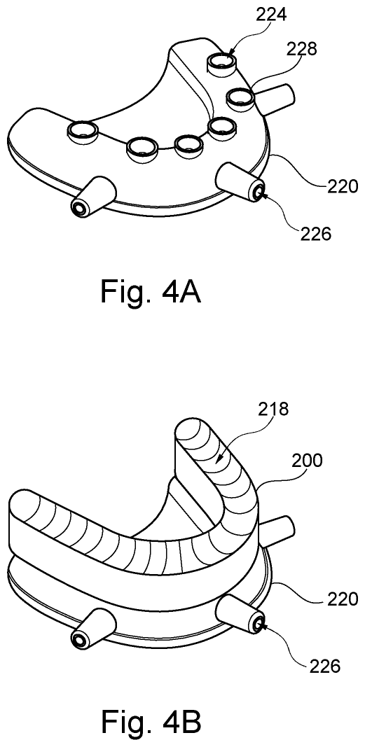

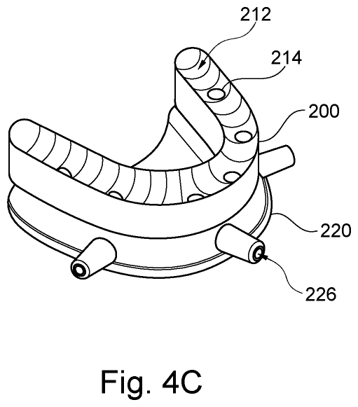

FIG. 4 show exemplary positioning elements and drilling templates,

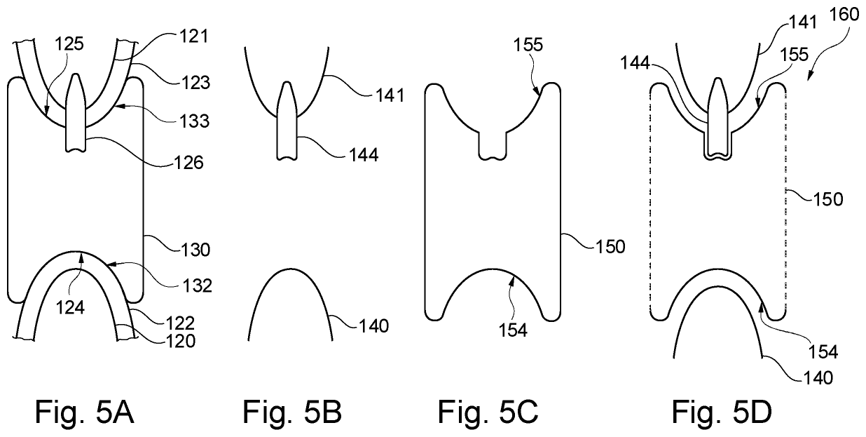

FIG. 5 show an exemplary bite register between jaws, as well as data of the jawbones and of the bite register,

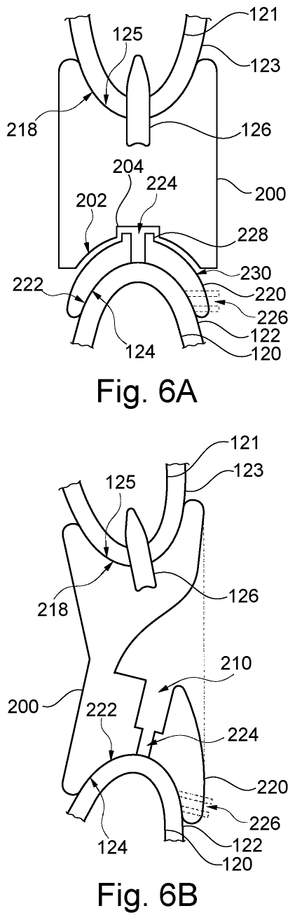

FIG. 6 show exemplary positioning elements and drilling templates,

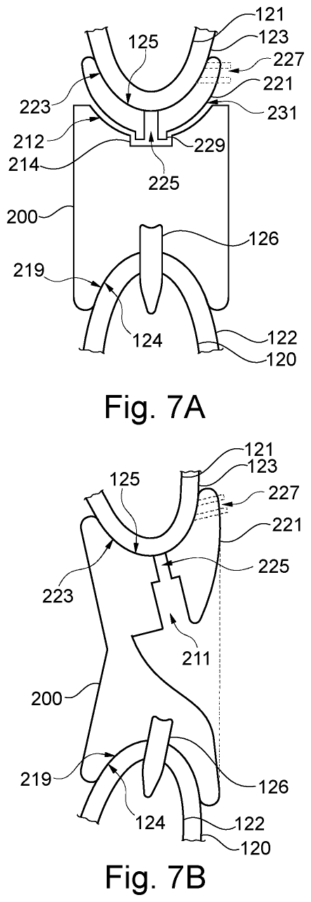

FIG. 7 show exemplary positioning elements and drilling templates,

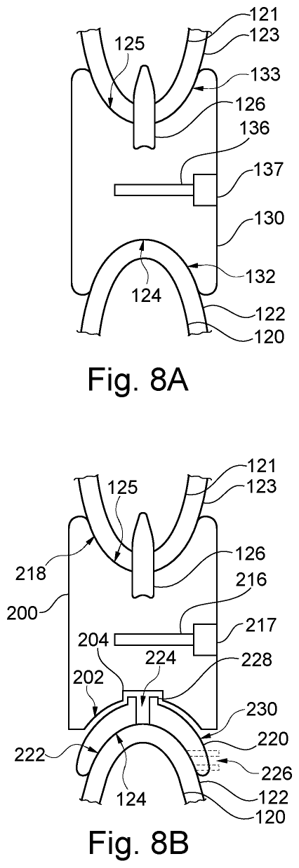

FIG. 8 show an exemplary bite register and an exemplary positioning element with pressure sensing elements,

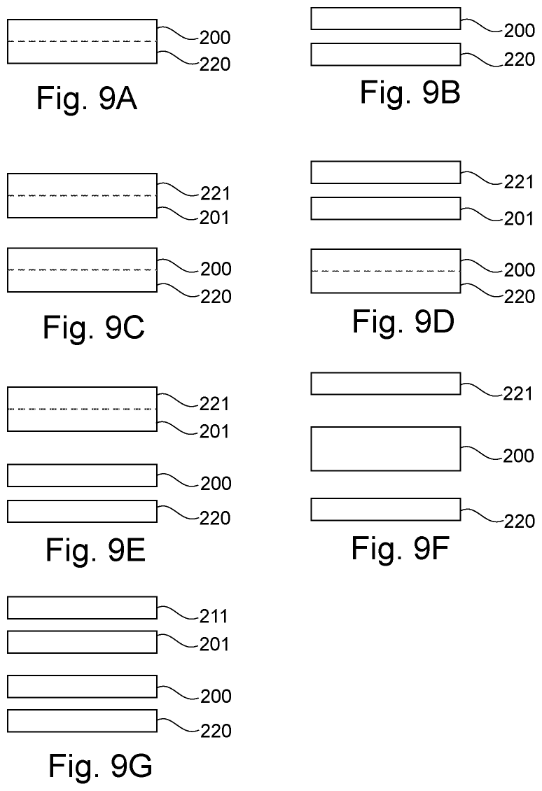

FIG. 9 show exemplary combinations of schematic positioning elements and drilling templates,

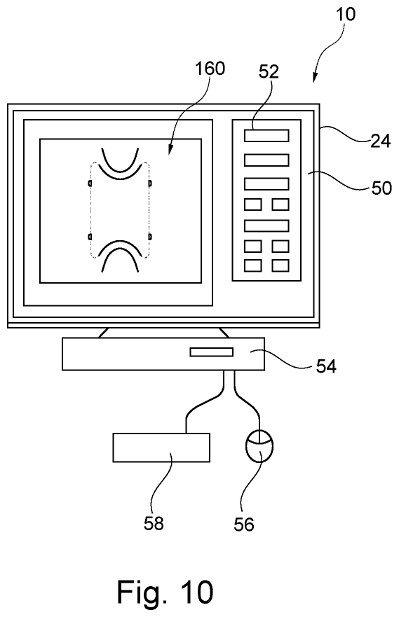

FIG. 10 shows an exemplary computer system for creating digital models of positioning elements and drilling templates,

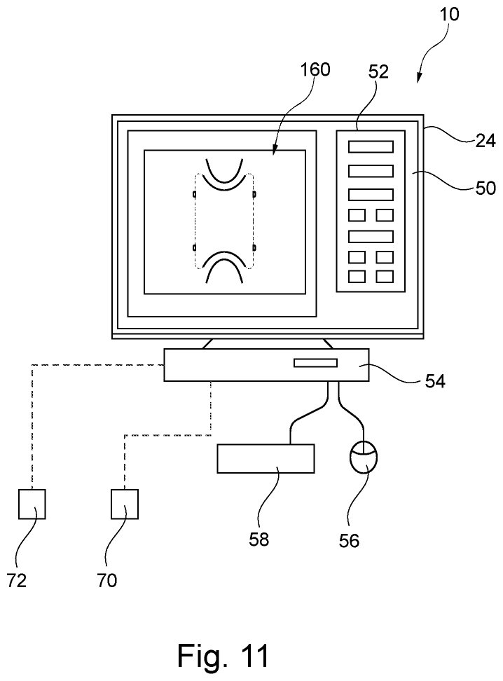

FIG. 11 shows an exemplary computer system for creating digital models of positioning elements and drilling templates,

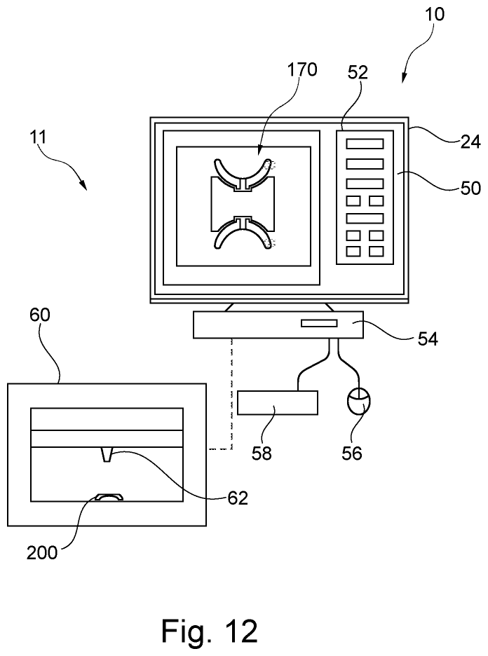

FIG. 12 shows an exemplary production device for producing positioning elements and drilling templates,

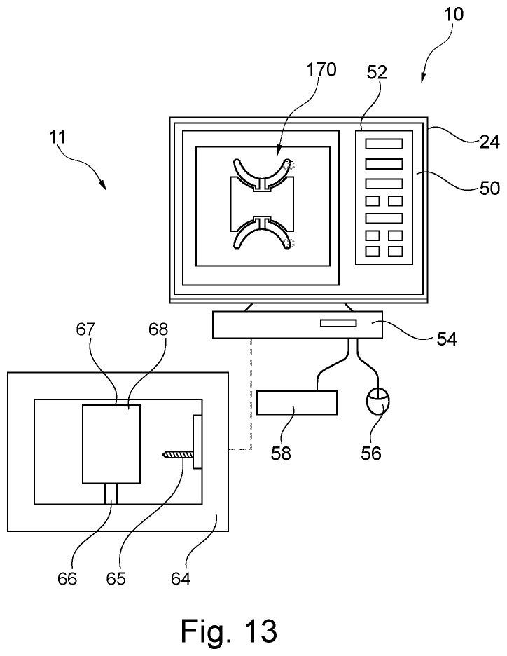

FIG. 13 shows an exemplary production device for producing positioning elements and drilling templates,

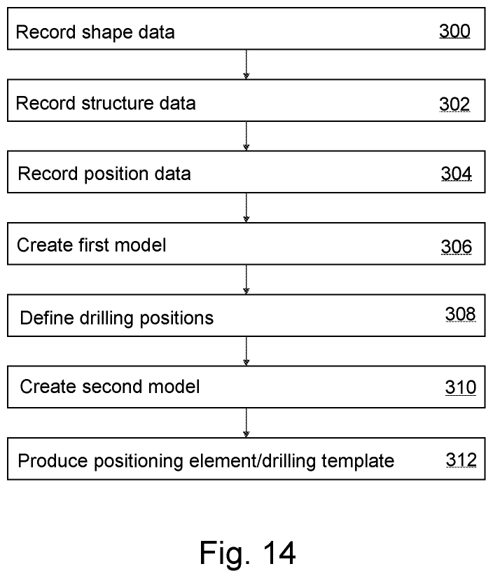

FIG. 14 shows a schematic flow diagram of an exemplary method for producing a positioning element, and

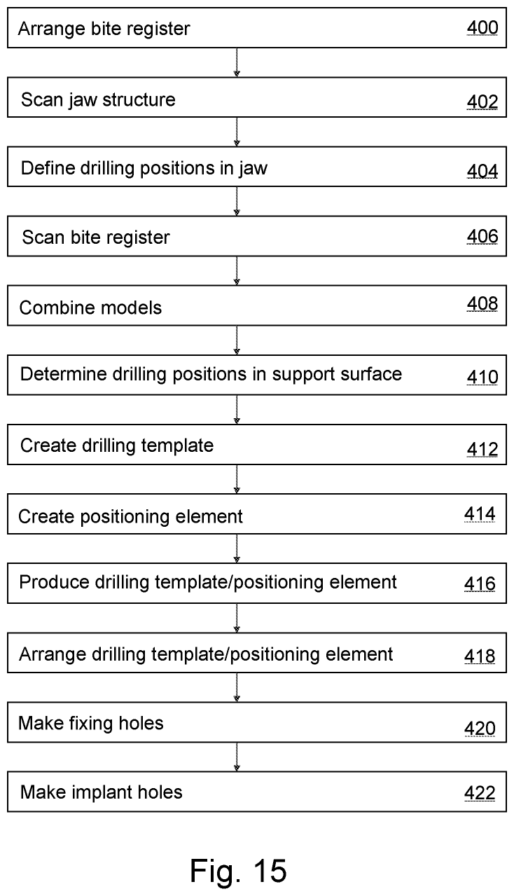

FIG. 15 shows a schematic flow diagram of an exemplary method for producing and using a positioning element.

Elements of the following examples that correspond to each other are denoted by the same reference signs.

FIGS. 1A to 1D show cross-sections of an exemplary bite register 130 between jaws, as well as data of the jawbones 140, 141 and of the bite register 150. FIG. 1A shows a cross-section through a jawbone 120 with a gingiva 122 arranged thereon, as well as through an opposing jawbone 121 and a gingiva 123 arranged thereon. Arranged between the two jaws is a bite register 130, which by way of example comprises markers 134. By way of example, the markers 134 are opaque, i.e. visible, for a scanning method for recording the structures of the two jawbones 120, 121. The patient bites on the bite register 123, as a result of which impressions 132, 133 are created in the bite register. The geometric shape of the impression 132 corresponds, for example, to the geometric shape of a surface 124 of the gingiva 122 of the jaw, i.e. is the negative thereof, while the geometric shape of the impression 133 corresponds, for example, to the geometric shape of a surface 125 of the gingiva 132 of the opposing jaw, i.e. is the negative thereof. By way of example, both jaws are toothless.

FIG. 1B shows the scan data from a scan of the structures of the jawbone 120 and opposing jawbone 121. Besides scan data 140 of the jawbone 120 and scan data 141 of the opposing jawbone 121, the scan data also comprise scan data 142 of the markers 134. Other parts of the bite register 130, or the entire bite register 130, could for example also be opaque, i.e. visible, for the scanning method for recording the structures of the two jawbones 120, 121. If other parts of the bite register 130 or the entire bite register 130 are opaque, FIG. 1B would additionally comprise scan data of such parts of the bite register 130 or of the entire bite register 130.

FIG. 1C shows scan data from a second scanning method for recording shape data of the bite register 130, in particular of the impressions 132, 133. The scan data 150 of the bite register comprise, for example, at least scan data 154, 155 of the impressions 132, 133 and scan data 152 of the markers 134. By way of example, the scan data 150 may also comprise the entire surface of the bite register 130. By way of example, if the entire bite register 130 is opaque for the scanning method for recording the structures of the two jawbones 120, 121, the second scanning method could even be omitted, since then the first scanning method could already comprise all the surface data of the bite register 130.

FIG. 1D shows a cross-section through a digital model 160 of the jawbones 120, 121 and of the bite register 130. The digital model 160 comprises the scan data 140 of the jawbone 120, the scan data 141 of the opposing jawbone 121 and the scan data 154, 155 of the impressions 132, 133. The position of the bite register 130 relative to the jawbones 120, 121 is determined by bringing into alignment the scan data 142, 152 of the markers 134 recorded by the two scanning methods. The scan data 142, 152 of the markers 134 therefore represent position data of the bite register 130 relative to the jawbones 120, 121. By way of example, the contours of the gingivae 122, 123 are also indirectly recorded through the scan data 154, 155 of the jawbone 140 and opposing jawbone 141 and the scan data 154, 155 of the impressions 132, 133, even though the gingivae 122, 123 are not directly recorded by the scanning methods, for example. By way of example, the scan data 154, 155 of the impressions 132, 133 are used as specifications for the geometric shape of template contact surfaces of drilling templates that are to be created. The position of the scan data 154, 155 of the impressions 132, 133 relative to the scan data 154, 155 of the jawbones 140, 141 defines, for example, a reference position for the template contact surfaces relative to the jawbones 140, 141.

FIGS. 2A to 2D show cross-sections of exemplary positioning elements and drilling templates. The positioning elements and drilling templates in question correspond to digital models of the positioning elements and drilling templates, which are created on a computer using the digital model 160. By way of example, the digital models of the positioning elements and drilling templates are adjusted to the geometric conditions in the patient's oral cavity, which are provided by the digital model 160. FIG. 2A shows two drilling templates 220, 221 which are arranged on the gingiva 122 of the jaw or jawbone 120 and on the gingiva 123 of the opposing jaw or opposing jawbone 121. The drilling templates 220, 221 each comprise a template contact surface 222, 223, the geometric shape of which is predefined by the impressions 132, 133 of the gingival surfaces 124 and 125 recorded using the bite register 130. By way of example, the drilling templates 220, 221 each comprise through-openings 224, 225 for making implant holes in the jawbones 120, 121 through the gingivae 122, 123. By way of example, the drilling templates 220, 221 each also comprise further through-openings 226, 227 for making fixing holes in the jawbones 120, 121 through the gingivae 122, 123. Fixing elements for fixing the drilling templates 220, 221 in the positions relative to the jawbones 120, 121 that are predefined by the positioning element 200 can be arranged in the fixing holes. By way of example, both jaws are toothless. The drilling templates 220, 221 are positioned and fixed, by using the positioning element 200 arranged therebetween, in positions relative to the jawbones 120, 121. The shape and dimensions of the positioning element 200 are determined such that the positions of the drilling templates 220, 221 or template contact surfaces 222, 223 relative to the jawbones 120, 121 correspond to the reference positions defined in FIG. 1D by the scan data of the impressions. By way of example, the shape and dimensions of the positioning element 200 are determined such that the positioning element 200 together with the drilling templates 220, 221 corresponds, in terms of dimensions, to the bite register 130 containing the impressions 132, 133. The positioning and fixing of the drilling templates 220, 221 by the positioning element 200 takes place, in a pressure-based manner, by pressure which the patient exerts on the positioning element 200 with his jaws via the drilling templates 220, 221.

By way of example, the positioning element 200 comprises contact surfaces 202, 212 for coming into conclusive contact with surfaces 230, 231 of the drilling templates 220, 221. By way of example, the positioning element 200 and the drilling templates 220, 221 comprise connecting elements for establishing a non-destructively releasable connection between the positioning element 200 and the respective drilling templates 220, 221. Furthermore, such connecting elements can also be used to align the positioning element 200 relative to the drilling templates 220, 221. By way of example, the positioning element 200 comprises female connecting elements 204, 214 which are intended to receive male connecting elements 228, 229 of the drilling templates 220, 221. In addition or as an alternative, the drilling templates 220, 221 could also comprise, for example, female connecting elements which are intended to receive male connecting elements of the positioning element 200.

Using the positioning element 200, the drilling templates 220, 221 can be positioned and fixed, in a pressure-based manner, in the intended positions for making fixing holes and inserting fixing elements. The drilling templates 220, 221 can be fixed at the intended positions, in a pressure-independent manner, by means of the fixing elements. By way of example, the patient can thus open the mouth and the positioning element 200 can be removed from the oral cavity. By way of example, removing the positioning element 200 enables access to the through-openings 224, 225 of the drilling templates 220, 221 in order to make the implant holes.

FIG. 2B shows two drilling templates 220, 221, which correspond to the drilling templates 220, 221 of FIG. 2A. FIG. 2B shows two positioning elements 200, 201, which can be connected to each other via a non-destructively releasable connection. The combination of the two positioning elements 200, 201 corresponds, for example, to the single positioning element 200 shown in FIG. 2A. Besides a contact surface 202 for coming into contact with the drilling template 220, the positioning element 200 shown in FIG. 2B also comprises, for example, a female connecting element 204 for receiving a male connecting element 228 of the drilling template 220. By way of example, the positioning element 200 also comprises connecting elements for establishing the non-destructively releasable connection to the positioning element 201. By way of example, the positioning element 200 comprises female connecting elements 208 for receiving male connecting elements 207 of the positioning element 201, and/or comprises male connecting elements 206 configured to be received in female connecting elements 209 of the positioning element 201. Besides a contact surface 203 for coming into contact with the drilling template 221, the positioning element 201 also comprises, for example, a female connecting element 205 for receiving a male connecting element 229 of the drilling template 221. By way of example, the positioning element 201 also comprises connecting elements for establishing the non-destructively releasable connection to the positioning element 200. By way of example, the positioning element 201 comprises female connecting elements 209 for receiving male connecting elements 206 of the positioning element 200, and/or comprises male connecting elements 207 configured to be received in female connecting elements 208 of the positioning element 200. The connecting elements can also be used to align the two positioning elements 200, 201 relative to each other.

Using the positioning elements 200, 201, the drilling templates 220, 221 can be positioned and fixed, in a pressure-based manner, in the intended positions for making fixing holes and inserting fixing elements. The drilling templates 220, 221 can be fixed at the intended positions, in a pressure-independent manner, by means of the fixing elements. By way of example, the patient can thus open the mouth and the positioning elements 200, 201 can be removed from the oral cavity. By way of example, removing the positioning element 200 enables access to the through-openings 224, 225 of the drilling templates 220, 221 in order to make the implant holes.

FIG. 2C shows an exemplary combination of two drilling templates 220, 221 and one positioning element 200. The positioning element 200 is comprised by the two drilling templates 220, 221. By way of example, the combination of the positioning element 200 and the two drilling templates 220, 221 is formed in one piece. The two drilling templates 220, 221 each comprise a template contact surface 222, 223. By way of example, the drilling templates 220, 221 each also comprise through-openings 224, 225 for making implant holes in the jawbones 120, 121 through the gingivae 122, 123. By way of example, the drilling templates 220, 221 may each also comprise further through-openings 226, 227 for making fixing holes in the jawbones 120, 121 through the gingivae 122, 123. Fixing elements for fixing the drilling templates 220, 221 in the positions relative to the jawbones 120, 121 that are predefined by the positioning element 200 can be inserted in the fixing holes. By way of example, the positioning element 200 comprises access openings 210, 211 which enable access to the through-openings 224, 225 of the drilling templates 220, 221 while the positioning element 200 is arranged in the patient's oral cavity. This means, for example, that the positioning element 200 does not have to be removed from the patient's oral cavity in order to make the implant holes. While implant holes are being made, the drilling templates 220, 221 can be fixed in the intended position, in a pressure-dependent manner, using the positioning element 200 and/or can be fixed in the intended position, in a pressure-independent manner, using fixing elements. In order to stabilize the positioning element 200, the positioning element may comprise additional support elements which extend between the drilling templates 220, 221. By way of example, the access openings 210, 211 are arranged between the support elements.

FIG. 2D shows two drilling templates 220, 221, which each comprise a positioning element 200, 201. By way of example, the drilling templates 220, 221 are each formed in one piece with the corresponding positioning element 200, 201. The two drilling templates 220, 221 each comprise a template contact surface 222, 223. By way of example, the drilling templates 220, 221 each also comprise through-openings 224, 225 for making implant holes in the jawbones 120, 121 through the gingivae 122, 123. By way of example, the drilling templates 220, 221 may each also comprise further through-openings 226, 227 for making fixing holes in the jawbones 120, 121 through the gingivae 122, 123. Fixing elements for fixing the drilling templates 220, 221 in the positions relative to the jawbones 120, 121 that are predefined by the positioning element 200 can be inserted in the fixing holes. By way of example, the positioning elements 200, 201 each comprise an access opening 210, 211 which enables access to the through-opening 224, 225 of the corresponding drilling template 220, 221 while the positioning elements 200, 201 are arranged in the patient's oral cavity. This means, for example, that the positioning elements 200, 201 do not have to be removed from the patient's oral cavity in order to make the implant holes.

By way of example, the positioning elements 200, 201 can be connected to each other via a non-destructively releasable connection. By way of example, the positioning element 200 comprises connecting elements for establishing the non-destructively releasable connection to the positioning element 201. By way of example, the positioning element 200 comprises female connecting elements 208 for receiving male connecting elements 207 of the positioning element 201, and/or comprises male connecting elements 206 configured to be received in female connecting elements 209 of the positioning element 201. By way of example, the positioning element 201 comprises connecting elements for establishing the non-destructively releasable connection to the positioning element 200. By way of example, the positioning element 201 comprises female connecting elements 209 for receiving male connecting elements 206 of the positioning element 200, and/or comprises male connecting elements 207 configured to be received in female connecting elements 208 of the positioning element 200. Furthermore, the connecting elements can also be used to align the two positioning elements 200, 201 relative to each other.

By way of example, in order to make the implant holes, the non-destructively releasable connection between the two positioning elements 200, 201 is released by opening the patient's jaws, and the two positioning elements 200, 201 are distanced from each other so as to enable access to the through-openings 224, 225 via the access openings 210, 211. The through-openings 224, 225 can thus be used to make the implant holes. While implant holes are being made, the drilling templates 220, 221 can be fixed in the intended position, in a pressure-independent manner, using fixing elements.

FIGS. 3A and 3B show cross-sections of an exemplary bite register 130 and of an exemplary positioning element 200 with pressure sensing elements 136, 216. FIG. 3A shows a bite register 130 which corresponds to the bite register 130 shown in FIG. 1A. The bite register 130 in FIG. 3A additionally has a pressure sensing element 136 for sensing a pressure that is applied to the bite register 130 by the jaws during the scanning of the jawbone structure data. The pressure sensing element 136 may be, for example, a piezoelectric sensor. The bite register 130 may further comprise a pressure indicating element 137 which indicates the pressure sensed by the pressure sensing element 136. By way of example, the pressure indicating element 137 may be comprised by the bite register 130 or may be provided as an external pressure indicating element 137. By way of example, the pressure indicating element 137 provides a visual indication of the pressure sensed by the pressure sensing element 136. By way of example, the visual indication may take place quantitatively in the form of numbers or qualitatively in the form of colors. By way of example, the pressure indicating element 137 may also be provided by the pressure sensing element 136, for instance in the case of a pressure sensing element 136 in the form of a pressure-dependent deformation element, the shape of which is pressure-dependent, or in the form of a pressure-dependent color element, the color of which is pressure-dependent. The sensed pressure applied to the bite register 130 by the jaws during the scanning of the jawbone structure data can be used as a reference pressure.

FIG. 3B shows a positioning element 200 which corresponds to the positioning element 200 shown in FIG. 2A. The positioning element 200 in FIG. 3B additionally has a pressure sensing element 216 for sensing a pressure that is applied to the positioning element 200 by the jaws. The pressure sensing element 216 may be, for example, a piezoelectric sensor. The positioning element 200 may further comprise a pressure indicating element 217 which indicates the pressure sensed by the pressure sensing element 216. By way of example, the pressure indicating element 217 may be comprised by the positioning element 200 or may be provided as an external pressure indicating element 217. By way of example, the pressure indicating element 217 provides a visual indication of the pressure sensed by the pressure sensing element 216. By way of example, the visual indication may take place quantitatively in the form of numbers or qualitatively in the form of colors. By way of example, the pressure indicating element 217 may also be provided by the pressure sensing element 216, for instance in the case of a pressure sensing element 216 in the form of a pressure-dependent deformation element, the shape of which is pressure-dependent, or in the form of a pressure-dependent color element, the color of which is pressure-dependent. By means of the pressure sensing element 216, it can be ensured that a pressure identical to the reference pressure is being applied to the positioning element 200. By way of example, the further exemplary positioning elements 200, 201 shown in FIGS. 2B to 2D may also comprise a pressure sensing element 216 and/or a pressure indicating element 217.

FIGS. 4A to 4C show exemplary positioning elements 200 and drilling templates 220. FIG. 4A shows a perspective view of a drilling template 220 which is configured to be arranged on a patient's jaw, for example a toothless jaw. The drilling template 220 comprises through-openings 224 for making implant holes at previously defined implant positions. The drilling template 220 further comprises connecting elements 228 for establishing a non-destructively releasable connection to the positioning element 200. The connecting elements 228 are shown by way of example in FIG. 4A as elevations around the mouths of the through-openings 224. However, they could also be provided independently of the through-openings 224. By way of example, the connecting elements 228 are intended to be received in receptacles of the positioning element 200, such as the connecting elements 214 shown in FIG. 4C. The elevations 228 may alternatively or additionally provide drilling guide elements. Furthermore, the mouths of the through-openings 224 in the drilling guide elements may each provide, for example, a stop for limiting the drilling depth. The drilling template 220 further comprises through-openings 226 for making fixing holes at previously defined fixing positions. These fixing holes are configured for the insertion of fixing elements, so as to be able to fix the drilling template 220 in an intended position on the jaw in a pressure-independent manner. The extensions on the drilling template 220, through which the through-openings 226 extend, provide drilling guide elements for example. Furthermore, the mouths of the through-openings 226 in the drilling guide elements may each provide, for example, a stop for limiting the drilling depth.

FIG. 4B shows the drilling template 220 of FIG. 4A with an exemplary positioning element 200 arranged thereon. The positioning element 200 comprises a jaw contact surface 218 for establishing contact with the surface of an opposing jaw. By way of example, a non-destructively releasable connection is established between the positioning element 200 and the drilling template 220. To this end, the positioning element 200 has, on a side facing toward the drilling template 220, a contact surface for establishing contact with the drilling template, which contact surface corresponds to the contact surface 212 shown in FIG. 4C. By way of example, the contact surface in question comprises connecting elements in the form of receptacles for receiving the connecting elements 228 of the drilling template 220.

FIG. 4C shows the drilling template 220 of FIG. 4A with an exemplary positioning element 200 arranged thereon. The positioning element 200 comprises a contact surface 212 for establishing contact with a further drilling template, which may for example be designed in the same way as the drilling template 220 shown in FIG. 4A. The contact surface 212 has connecting elements 214 for establishing a non-destructively releasable connection to the further drilling template. To this end, the connecting elements 214 are formed, for example, as receptacles for receiving connecting elements of the further drilling template, such as the connecting elements 228 of the drilling template 220 shown in FIG. 4A.

FIG. 5A to 5D show cross-sections of an exemplary bite register 130 between jaws, as well as data of the jawbones 140, 141 and of the bite register 150. FIG. 5A shows a cross-section through a jawbone 120 with a gingiva 122 arranged thereon, as well as through an opposing jawbone 121 and a gingiva 123 arranged thereon. Arranged between the two jaws is a bite register 130, which corresponds to the bite register shown in FIG. 1A but does not comprise any markers. In the case of FIG. 5A, the opposing jaw has, for example, a dental object 126, such as a tooth or an existing implant. The impression 133 therefore comprises, in addition to an impression of the gingival surface 125, also an impression of the dental object 126. By way of example, the dental object 126 is a fixed object which can be used as a reference structure. By way of example, the dental object 126 is opaque for the scanning method for recording the jawbone structure data. At the same time, the matching impression of the dental object 126 can be recorded by the second scanning method for recording the shape data of the impression. The shape data of the impression form the matching negative to the dental object 126. By bringing the shape data of the impression of the dental object 126 into alignment with the structure data of the dental object 126, the position of data of the bite register 130 relative to data of the dental object 126 can be determined. Therefore, additional markers may be superfluous.

FIG. 5B shows scan data of a scan of the structures of the jawbone 120 and opposing jawbone 121. Besides scan data 140 of the jawbone 120 and scan data 141 of the opposing jawbone 121, the scan data also comprise scan data 144 of the dental object 126. FIG. 5C shows scan data of a second scanning method for recording shape data of the bite register 130, in particular of the impressions 132, 133. By way of example, the scan data 150 of the bite register comprise at least scan data 154, 155 of the impressions 132, 133, wherein the impression 133 comprises an impression of the dental object 126. By way of example, the scan data 150 may also comprise the entire surface of the bite register 130.

FIG. 5D shows a cross-section through a digital model 160 of the jawbones 120, 121 and of the bite register 130. The digital model 160 comprises the scan data 140 of the jawbone 120, the scan data 141 of the opposing jawbone 121, the scan data 144 of the dental object 126 and the scan data 154, 155 of the impressions 132, 133. The position of the bite register 130 relative to the jawbones 120, 121 is determined by bringing the scan data 144 of the dental object 126, recorded by the first scanning method, into alignment with the scan data of the impression 155, recorded by the second scanning method, which impression comprises an impression of the dental object 126. The scan data 144 of the dental object 126 together with the scan data 155 of the impression of the dental object 126 therefore represent position data of the bite register 130 relative to the jawbones 120, 121. By way of example, the contours of the gingivae 122, 123 are also indirectly recorded through the scan data 154, 155 of the jawbone 140 and the opposing jawbone 141 and the scan data 154, 155 of the impressions 132, 133, even though the gingivae 122, 123 are not directly recorded by the scanning methods, for example. By way of example, the scan data 154 of the impression 132 are used as a specification for a geometric shape of a template contact surface of a drilling template that is to be created. By way of example, the position of the scan data 154, 155 of the impressions 132, 133 relative to the scan data 154, 155 of the jawbones 140, 141 defines a reference position for the template contact surface of the drilling template relative to the jawbones 140, 141.

FIGS. 6A and 6B show cross-sections of exemplary positioning elements 200 and drilling templates 220. FIG. 6A shows a drilling template 220 which is arranged on the gingiva 122 of the jaw or jawbone 120. The drilling template 220 comprises a template contact surface 222, the geometric shape of which is predefined by the impression 132 of the gingival surfaces 124 recorded using the bite register 130. By way of example, the drilling template 220 comprises through-openings 224 for making implant holes in the jawbone 120 through the gingiva 122. By way of example, the drilling templates 220 each also comprise further through-openings 226 for making fixing holes in the jawbone 120 through the gingiva 122. Fixing elements for fixing the drilling templates 220 in the position relative to the jawbone 120 that is predefined by the positioning element 200 can be arranged in the fixing holes. The drilling template 220 is positioned and fixed in a position relative to the jawbone 120 using the positioning element 200 arranged between the drilling template 220 and the gingiva 123 of the opposing jawbone 121. The shape and dimensions of the positioning element 200 are determined such that the position of the drilling templates 220 or template contact surface 222 relative to the jawbone 120 corresponds to the reference position defined by the scan data 154 of the impression 132 in FIG. 5D. By way of example, the shape and dimensions of the positioning element 200 are determined such that the positioning element 200 together with the drilling template 220 corresponds, in terms of dimensions, to the bite register 130 containing the impressions 132, 133. The positioning and fixing of the drilling template 220 by the positioning element 200 takes place, in a pressure-based manner, by pressure which the patient exerts on the drilling template 220 and the positioning element 200 with his jaws.

By way of example, the positioning element 200 comprises a contact surface 202 for coming into conclusive contact with the surfaces 230 of the drilling template 220. By way of example, the positioning element 200 and the drilling template 220 comprise connecting elements for establishing a non-destructively releasable connection between the positioning element 200 and the drilling templates 220. Furthermore, such connecting elements can also be used to align the positioning element 200 relative to the drilling templates 220. By way of example, the positioning element 200 comprises female connecting elements 204 which are intended to receive male connecting elements 228 of the drilling template 220. In addition or as an alternative, the drilling template 220 could also comprise, for example, female connecting elements which are intended to receive male connecting elements of the positioning element 200.

By way of example, the positioning element 200 further comprises a jaw contact surface 218 for coming into contact with the surfaces 125 of the gingiva 123 and the dental object 126 of the opposing jaw. As a result of contact with the dental object 126, the positioning element 200 can be aligned relative to the opposing jawbone 121. The geometric shape of the jaw contact surface 218 is defined, for example, by the scan data 155 of the impression 133 containing the impression of the dental object 126.

Using the positioning element 200, the drilling template 220 can be positioned and fixed, in a pressure-based manner, in the intended position for making fixing holes and inserting fixing elements. The drilling template 220 can be fixed at the intended position, in a pressure-independent manner, by means of the fixing elements. By way of example, the patient can thus open the mouth and the positioning element 200 can be removed from the oral cavity. By way of example, removing the positioning element 200 enables access to the through-openings 224 of the drilling template 220 in order to make the implant holes.

FIG. 6B shows an exemplary combination of a drilling template 220 and a positioning element 200. The positioning element 200 is comprised by the drilling template 220. By way of example, the combination of the positioning element 200 and the drilling template 220 is formed in one piece. The drilling template 220 comprises a template contact surface 222. By way of example, the drilling template 220 also comprises through-openings 224 for making implant holes in the jawbone 120 through the gingiva 122. By way of example, the drilling template 220 may also comprise further through-openings 226 for making fixing holes in the jawbone 120 through the gingiva 122. Fixing elements for fixing the drilling template 220 in the position relative to the jawbone 120 that is predefined by the positioning element 200 can be arranged in the fixing holes. By way of example, the positioning element 200 comprises access openings 210 which enable access to the through-openings 224 of the drilling template 220 while the positioning element 200 is arranged in the patient's oral cavity. This means, for example, that the positioning element 200 does not have to be removed from the patient's oral cavity in order to make the implant holes. While implant holes are being made, the drilling template 220 can be fixed in the intended position, in a pressure-dependent manner, using the positioning element 200 and/or can be fixed in the intended position, in a pressure-independent manner, using fixing elements. In order to stabilize the positioning element 200, the positioning element may comprise additional support elements. By way of example, the access openings 210 are arranged between the support elements.

By way of example, the positioning element 200 further comprises a jaw contact surface 218 for coming into contact with the surfaces 125 of the gingiva 123 and the dental object 126 of the opposing jaw. As a result of contact with the dental object 126, the positioning element 200 can be aligned relative to the opposing jawbone 121. The geometric shape of the jaw contact surface 218 is defined, for example, by the scan data 155 of the impression 133 containing the impression of the dental object 126.

FIGS. 7A and 7B show cross-sections of exemplary positioning elements 200 and drilling templates 221. FIG. 7A shows a situation which corresponds to FIG. 6A. However, it is the jaw and not the opposing jaw that has the dental object 126, and the drilling template 221 is arranged on the gingiva 123 of the other jaw or jawbone 121. The drilling template 221 comprises a template contact surface 223, the geometric shape of which is predefined by the impression 133 of the gingival surfaces 125 recorded using the bite register 130. By way of example, the drilling template 220 comprises through-openings 225 for making implant holes in the jawbone 121 through the gingiva 123. By way of example, the drilling templates 221 each also comprise further through-openings 227 for making fixing holes in the jawbone 121 through the gingiva 123. Fixing elements for fixing the drilling templates 221 in the position relative to the jawbone 121 that is predefined by the positioning element 200 can be arranged in the fixing holes. The drilling template 221 is positioned and fixed in a position relative to the jawbone 121 using the positioning element 200 arranged between the drilling template 221 and the gingiva 122 of the jawbone 120. The positioning and fixing of the drilling template 221 by the positioning element 200 takes place, in a pressure-based manner, by pressure which the patient exerts on the drilling template 221 and the positioning element 200 with his jaws.

By way of example, the positioning element 201 comprises a contact surface 212 for coming into conclusive contact with the surfaces 231 of the drilling template 221. By way of example, the positioning element 200 and the drilling template 221 comprise connecting elements for establishing a non-destructively releasable connection between the positioning element 200 and the drilling templates 221. Furthermore, such connecting elements can also be used to align the positioning element 200 relative to the drilling templates 221. By way of example, the positioning element 200 comprises female connecting elements 214 which are intended to receive male connecting elements 229 of the drilling template 221. In addition or as an alternative, the drilling template 221 could also comprise, for example, female connecting elements which are intended to receive male connecting elements of the positioning element 200.

By way of example, the positioning element 200 further comprises a jaw contact surface 219 for coming into contact with the surfaces 124 of the gingiva 122 and the dental object 126. As a result of contact with the dental object 126, the positioning element 200 can be aligned relative to the jawbone 120. The geometric shape of the jaw contact surface 219 is defined, for example, by scan data of an impression of the bite register containing the impression of the dental object 126.

Using the positioning element 200, the drilling template 221 can be positioned and fixed, in a pressure-based manner, in the intended position for making fixing holes and inserting fixing elements. The drilling template 221 can be fixed at the intended position, in a pressure-independent manner, by means of the fixing elements. By way of example, the patient can thus open the mouth and the positioning element 200 can be removed from the oral cavity. By way of example, removing the positioning element 200 enables access to the through-openings 225 of the drilling template 221 in order to make the implant holes.

FIG. 7B shows a situation which corresponds to FIG. 6B. However, it is the jaw and not the opposing jaw that has the dental object 126, and the drilling template 221 is arranged on the gingiva 123 of the other jaw or jawbone 121. FIG. 7B shows an exemplary combination of a drilling template 221 and a positioning element 200. The positioning element 200 is comprised by the drilling template 221. By way of example, the combination of the positioning element 200 and the drilling template 221 is formed in one piece. The drilling template 221 comprises a template contact surface 223. Byway of example, the drilling template 221 also comprises through-openings 225 for making implant holes in the jawbone 121 through the gingiva 123. By way of example, the drilling template 221 may also comprise further through-openings 227 for making fixing holes in the jawbone 121 through the gingiva 123. Fixing elements for fixing the drilling template 221 in the position relative to the jawbone 121 that is predefined by the positioning element 200 can be arranged in the fixing holes. By way of example, the positioning element 200 comprises access openings 211 which enable access to the through-openings 225 of the drilling template 221 while the positioning element 200 is arranged in the patient's oral cavity. This means, for example, that the positioning element 200 does not have to be removed from the patient's oral cavity in order to make the implant holes. While implant holes are being made, the drilling template 221 can be fixed in the intended position, in a pressure-dependent manner, using the positioning element 200 and/or can be fixed in the intended position, in a pressure-independent manner, using fixing elements. In order to stabilize the positioning element 200, the positioning element may comprise additional support elements. By way of example, the access openings 211 are arranged between the support elements.

By way of example, the positioning element 200 further comprises a jaw contact surface 219 for coming into contact with the surfaces 124 of the gingiva 122 and the dental object 126. As a result of contact with the dental object 126, the positioning element 200 can be aligned relative to the opposing jawbone 120. The geometric shape of the jaw contact surface 219 is defined, for example, by scan data of an impression of a bite register containing the impression of the dental object 126.

FIGS. 8A and 8B show cross-sections of an exemplary bite register 130 and of an exemplary positioning element 200 with pressure sensing elements 136, 216. FIG. 8A shows a bite register 130 which corresponds to the bite register 130 shown in FIG. 5A. The bite register 130 in FIG. 8A additionally has a pressure sensing element 136 for sensing a pressure that is applied to the bite register 130 by the jaws during the scanning of the jawbone structure data. The pressure sensing element 136 may be, for example, a piezoelectric sensor. The bite register 130 may further comprise a pressure indicating element 137 which indicates the pressure sensed by the pressure sensing element 136. By way of example, the pressure indicating element 137 may be comprised by the bite register 130 or may be provided as an external pressure indicating element 137. By way of example, the pressure indicating element 137 provides a visual indication of the pressure sensed by the pressure sensing element 136. By way of example, the visual indication may take place quantitatively in the form of numbers or qualitatively in the form of colors. By way of example, the pressure indicating element 137 may also be provided by the pressure sensing element 136, for instance in the case of a pressure sensing element 136 in the form of a pressure-dependent deformation element, the shape of which is pressure-dependent, or in the form of a pressure-dependent color element, the color of which is pressure-dependent. The sensed pressure applied to the bite register 130 by the jaws during the scanning of the jawbone structure data can be used as a reference pressure.

FIG. 8B shows a positioning element 200 which corresponds to the positioning element 200 shown in FIG. 6A. The positioning element 200 in FIG. 8B additionally has a pressure sensing element 216 for sensing a pressure that is applied to the positioning element 200 by the jaws. The pressure sensing element 216 may be, for example, a piezoelectric sensor. The positioning element 200 may further comprise a pressure indicating element 217 which indicates the pressure sensed by the pressure sensing element 216. By way of example, the pressure indicating element 217 may be comprised by the positioning element 200 or may be provided as an external pressure indicating element 217. By way of example, the pressure indicating element 217 provides a visual indication of the pressure sensed by the pressure sensing element 216. By way of example, the visual indication may take place quantitatively in the form of numbers or qualitatively in the form of colors. By way of example, the pressure indicating element 217 may also be provided by the pressure sensing element 216, for instance in the case of a pressure sensing element 216 in the form of a pressure-dependent deformation element, the shape of which is pressure-dependent, or in the form of a pressure-dependent color element, the color of which is pressure-dependent. By means of the pressure sensing element 216, it can be ensured that a pressure identical to the reference pressure is being applied to the positioning element 200. By way of example, the further exemplary positioning elements 200 shown in FIGS. 6B, 7A and 7B may also comprise a pressure sensing element 216 and/or a pressure indicating element 217.

FIGS. 9A to 9F show exemplary combinations of schematic positioning elements and drilling templates. FIG. 9A shows a drilling template 220 which comprises a positioning element 200. The drilling template 220 is configured, for example, to be positioned and fixed on a jaw using the positioning element 200. The positioning element 200 comprises, for example, a jaw contact surface for establishing contact with the surface of the opposing jaw.

FIG. 9B shows a drilling template 220 and a positioning element 200, which can be connected to each other by means of a non-destructively releasable connection. The drilling template 220 is configured, for example, to be positioned and fixed on a jaw using the positioning element 200. The positioning element 200 comprises, for example, a jaw contact surface for establishing contact with the surface of the opposing jaw.

FIG. 9C shows a first drilling template 220 which is configured, for example, to be positioned and fixed on a jaw using a first positioning element 200 comprised by the first drilling template 220. Also shown is a second drilling template 221 which is configured, for example, to be positioned and fixed on an opposing jaw using a second positioning element 201 comprised by the first drilling template 221. By way of example, the two positioning elements 200 and 201 can be connected to each other by means of a non-destructively releasable connection.

FIG. 9D shows a first drilling template 220 which is configured, for example, to be positioned and fixed on a jaw using a first positioning element 200 comprised by the first drilling template 220. Also shown is a second drilling template 221 and a second positioning element 201, which can be connected to each other by means of a non-destructively releasable connection. The second drilling template 221 is configured, for example, to be positioned and fixed on an opposing jaw using the second positioning element 201.

FIG. 9E shows a first drilling template 220 and a first positioning element 200, which can be connected to each other by means of a non-destructively releasable connection. The first drilling template 220 is configured, for example, to be positioned and fixed on a jaw using the first positioning element 200. Also shown is a second drilling template 221 which is configured, for example, to be positioned and fixed on an opposing jaw using a second positioning element 201 comprised by the first drilling template 221. By way of example, the two positioning elements 200 and 201 can be connected to each other by means of a non-destructively releasable connection.

FIG. 9F shows a first drilling template 220 and a first positioning element 200, which can be connected to each other by means of a non-destructively releasable connection. The first drilling template 220 is configured, for example, to be positioned and fixed on a jaw using the first positioning element 200. Also shown is a second drilling template 221 and a second positioning element 201, which can be connected to each other by means of a non-destructively releasable connection. The second drilling template 221 is configured, for example, to be positioned and fixed on an opposing jaw using the second positioning element 201.

FIG. 10 shows an exemplary computer system 10 for creating digital models of positioning elements and drilling templates. To create the digital models of positioning elements and drilling templates, use is made, for example, of a digital model 160 of jawbones and gingival surfaces. The digital model 160 is based, for example, on scans of jawbone structure data, shape data of a bite register, and position data of the position of the bite register relative to the jaws. By way of example, the computer system 10 may comprise a hardware component 54 having one or more processors, as well as a memory for storing machine-executable program commands. Execution of the program commands by the processors can cause the one or more processors to control the computer system 10 to construct the digital model 160 using scan data. The computer system 10 may further comprise external input devices, such as a keyboard 58 and a mouse 56, which enable the user to interact with the computer system 10. In addition, the computer system 10 may comprise output devices, such as a screen 24 comprising a user interface 50 with control elements 52, which enable the user to use the computer system 10 to control the construction of the digital model 160. The generated digital model 160 can be displayed on the user interface 50.

FIG. 11 shows an exemplary computer system 10 for creating digital models of positioning elements and drilling templates. The computer system 10 of FIG. 11 corresponds to the computer system 10 shown in FIG. 10. Compared to the computer system 10 shown in FIG. 10, the computer system 10 of FIG. 11 comprises a first and/or a second scanning device 70, 72. By way of example, the first scanning device 70 is configured to record structure data of jawbones in order to create the digital model 160. By way of example, the first scanning device 70 comprises a computed tomography device, a volumetric tomography device, or a magnetic resonance imaging device. By way of example, the second scanning device 72 is configured to record shape data of a bite register in order to create the digital model 160. By way of example, the second scanning device 72 comprises an optical scanner, a computed tomography device, or a haptic or tactile scanner.

FIG. 12 shows an exemplary production device 11 for producing positioning elements and drilling templates. The production device 11 may comprise, for example, the computer system 10 of FIG. 10. The computer system 10 may further be configured such that it controls a 3D printer 60 for producing positioning elements and drilling templates according to a digital model 170 constructed on the computer system 10. By way of example, the positioning element 200 may be printed. The 3D printer 60 may comprise a printing element 62 which is configured such that it prints positioning elements and drilling templates, for instance a positioning element 200, in a layer-by-layer manner. Depending on the embodiment, the computer system 10 may be configured to additionally use one or more further 3D printers 60.

FIG. 13 shows an exemplary production device 11 for producing positioning elements and drilling templates. The production device 11 may comprise, for example, the computer system 10 of FIG. 10. The computer system 10 may further be configured such that it controls a machining device 64 which is configured to use one or more machining tools 65 to machine a blank 67. The blank 67 made of material 68 may be provided by using a holding device 66 and may be cut or milled into a desired final shape and size of the element to be produced. By way of example, the one or more machining tools 72 are used to execute a material removal process controlled by the computer system 10. By way of example, positioning elements and/or drilling templates can be produced from the blank according to a digital model provided by the computer 10. By way of example, the machining tool 65 may be a milling tool. Depending on the embodiment, the computer system 10 may be configured to additionally use one or more further machining devices 64 and/or one or more 3D printers 60.

FIG. 14 shows a flow diagram of an exemplary method for producing a positioning element and/or a drilling template. The positioning element is configured to fix a drilling template on a gingiva of a patient's jaw. In this case, the positioning element fixes the drilling template, by means of pressure, which the patient applies to the positioning element by closing the jaw and an opposing jaw, in a position relative to a jawbone of the jaw for making implant holes in the jaw for dental implants. By way of example, the drilling template comprises a template contact surface for establishing contact with a surface of the gingiva of the jaw, as well as through-openings which each define an implant position for making one of the implant holes.

In block 300, shape data of an impression of the surface of the gingiva of the jaw are recorded using a bite register arranged between the patient's two jaws. During this, the two jaws are located in a recording position relative to each other, while the impression is located in a position relative to the jawbone which provides a reference position for the template contact surface defined by the impression.

In block 302, structure data of the jawbone are recorded while the bite register is arranged between the two jaws in the recording position. In block 304, position data of the bite register relative to the jawbone are recorded while the bite register is arranged between the two jaws in the recording position. By way of example, the recording of the jawbone structure data in block 302 and the recording of the position data in block 304 takes place simultaneously in the course of a scanning method. By way of example, the shape data in block 300 can be recorded in the course of the same scanning method if the bite register is opaque for the scanning method in question. If the bite register is not opaque for the scanning method in question, the recording of the shape data takes place, for example, in an extra step using a second scanning method. By way of example, the bite register is arranged between the patient's jaws and impressions of the jaw surfaces are created in the bite register. Blocks 302 and 304 can be executed while the bite register is arranged between the two jaws. The bite register containing the impressions can then be removed from the patient's oral cavity, and the shape data of the impression(s) can be recorded according to block 300 using the second scanning method.

In block 306, a first three-dimensional digital model of the jawbone structure and of the gingival surface is created using the data recorded in blocks 300, 302 and 304, i.e. the shape data, structure data and position data. In block 308, first implant positions for making the first implant holes in the jaw, i.e. through the gingiva into the jawbone, are defined using the model created in block 306. By way of example, digital models of the implant holes are added to the first model. In block 310, a second three-dimensional digital model of the drilling template(s) and of the positioning element(s) is created using the first model. The implant positions defined in block 308 are also taken into account. The shape and dimensions of the positioning elements are determined such that, when pressure is applied to the positioning element(s), the two jaws are arranged in the recording position. Furthermore, the shape and dimensions are determined such that the first drilling template(s) are arranged in a position relative to the jawbone that corresponds to the reference position.

By way of example, through-openings, through which the implant holes can be made in the jaw, are provided in the drilling template at each of the defined implant positions. Fixing positions for making fixing holes in the jaw, i.e. through the gingiva into the jawbone, can also be defined in block 308 in addition to the implant positions. By way of example, digital models of the fixing holes are added to the first model. The fixing holes are used to insert fixing elements through the drilling template(s) into the jaws or jawbones. The fixing elements are configured to fix the respective drilling template, in a jaw pressure-independent manner, in the reference position relative to the jaw or opposing jaw in order to make the implant holes. By way of example, in block 310, further through-openings, through which the fixing elements can be inserted in the jaw, are also provided in the drilling template at each of the defined fixing positions.

In block 312, the positioning elements and/or the drilling templates are produced using the second model. By way of example, production takes place using a computer-controlled production device, such as a CAM device. The production device comprises, for example, a 3D printer for printing positioning elements and/or drilling templates. In addition or as an alternative, the production device may provide, for example, a machining device for machining a blank, from which positioning elements and/or drilling templates are machined by means of a material-removing machining process.

FIG. 15 shows a flow diagram of an exemplary method for producing and using a positioning element and a drilling template. By way of example, in block 400, a bite register is arranged in the patient's mouth and an impression is made. By way of example, this impression is an impression of the gingiva of one jaw or two jaws of the patient. In the case of one jaw, this is, for example, the upper jaw or lower jaw of the patient. The jaws from which an impression is taken may be partially or entirely toothless. If the jaw is entirely toothless, the impression comprises, for example, only the surface of the gingiva. If the jaw is partially toothless, the impression comprises, for example, the one or more teeth in addition to the surface of the gingiva. In addition, the impression may optionally comprise dental prostheses arranged in or on the jaw. The impression may be, for example, an impression of the entire jaw or of a portion of the jaw.

By way of example, the bite register has a height such that the distance between the jaws when biting on the bite register corresponds approximately to the distance between the jaws with the future dental prosthesis. By way of example, the bite register has a height such that, when bit with a toothless jaw, it compensates for the height of the missing row of teeth of the toothless jaw. By way of example, the bite register has a height such that, when bit with two toothless jaws, it compensates for the height of the two missing rows of teeth of the toothless jaws. By way of example, the drilling template could be used to insert implants for a dental prosthesis in addition to an existing dental prosthesis. By way of example, the patient wears an existing dental prosthesis during the creation of the bite register. By way of example, the bite register has a height such that the distance between the jaws when biting on the bite register is greater than the distance between the jaws with the future dental prosthesis. By way of example, the height is selected such that the distance between jaws enables a dentist to make implant holes in one or both jaws.

By way of example, it can be ensured that, when biting on the bite register, a jaw position is achieved in which the bite register covers and/or contacts as fully as possible the area of the gingiva on which the drilling template to be created is to be arranged. This could have the advantage that the shape and/or structure of the surface of the area of the gingiva in question can be precisely recorded. Precise recording of the shape and/or structure of the surface makes it possible to create a drilling template with a template contact surface which sits firmly and precisely on the gingiva.

By way of example, the patient presses with his jaws on the bite register with a constant low bite force. In this case, the bite register could comprise a pressure or force gauge, which measures the bite force or the applied pressure. The patient could be given feedback, for example in optical or acoustic form, which informs the patient as soon as a predetermined bite force is reached.

By way of example, the bite register comprises a plurality of markers which are opaque, for instance radio-opaque, for a scanning method used to record the structure data of the jawbone. By way of example, the markers may be applied to the bite register, and/or parts of the bite register itself may be configured as markers, i.e. may be opaque. By way of example, apart from the markers, the bite register is transparent for the scanning method in question. This could have the advantage that, during the recording of the structure data, the position of the bite register relative to the jaw can be determined using the markers, which are visible in a scan of the structure data of the jaw. The fact that the bite register is otherwise transparent could also have the advantage that the structure data of the jaw are not obscured by the bite register during the scanning.

By way of example, a scanning method for which the gingiva of the jaw in question is transparent, i.e. invisible, is used to record the structure data of the jawbone. The bite register does not sit directly on the jawbone, but instead sits on the gingiva and is therefore distanced from the jawbone depending on the nature of the gingiva. Therefore, in the case of a bite register which is transparent for the scanning method in question, the position of the bite register relative to the jawbone cannot be determined from the scan data during the recording of the structure data. This is particularly true for toothless jaws, in the case of which, for example, only the jawbone structure is visible in such scan. By using suitable markers, for instance three or more, the position of the bite register relative to the jaw could be precisely determined. In turn, the position of the impression of the surface of the gingiva relative to the markers can be determined using the bite register itself, for instance using a second scanning method. By way of example, once the relative positions of markers to jaw and markers to surface of the gingiva are known, the position of the surface of the gingiva relative to the jawbone can be determined therefrom during the recording of the structure data, even if the gingiva itself is transparent for the scanning method used to record the structure data.

By way of example, both jaws are toothless, and therefore no fixed reference points, such as teeth, for determining the position of the bite register or of the impression of the bite register relative to the jaw can be detected in the course of a jawbone scanning method. Since the gingiva itself comprises only soft tissue, the yielding nature thereof means that it cannot provide suitable reference points for precise position determination. In other words, if a position of the surface of the gingiva relative to the jaw is determined independently of biting on the bite register, the varying pressure conditions would mean that no precise conclusions about the position of the gingival surface relative to the jaw can be drawn during the recording of the structure data.

By way of example, the markers are additionally configured to be detected by means of a further scanning method, for instance a haptic or optical scanning method. By way of example, this further scanning method is used to record impressions of the bite register, in particular of the surface of the gingiva.

If the jaw or the opposing jaw contains dental objects, such as teeth, which leave an impression in the bite register and are opaque for the scanning method used, these dental objects can be used as reference structures. Byway of example, other reference objects, such as an existing dental prosthesis, are present, which leave an impression in the bite register and are opaque for the scanning method used. This could have the advantage that the position of the gingival surface relative to the jawbone can be determined from the position of the corresponding reference points or reference objects relative to the jawbone and from the position of the impression of such reference points or reference objects relative to the impression of the surface of the gingiva. If such reference points or reference objects are present, the use of markers could be unnecessary, or the number of markers required could be reduced, for example to two or one. By way of example, such reference points or reference objects could be used to check a marker-based position determination, or vice versa.

By way of example, in block 402, the bone structure of the jaw is scanned while the bite register is arranged in the mouth. For the scan of the bone structure, a scanning power is used which is set such as to detect the density of the bones as precisely as possible.

By way of example, a representation (D1) of the patient's jaw or jaws is provided as a result of the scan. By way of example, the representation D1 is a three-dimensional digital model of the bone structure of the jaw.

By way of example, the DICOM standard is used to provide the representation D1. DICOM (“Digital Imaging and Communications in Medicine”) is an open standard for storing and exchanging information in medical image data management. This information may be, for example, digital images, additional information such as segmentations, surface definitions, etc., or image registrations. DICOM standardizes both the format for storing the data and also the communication protocol for exchanging the data.

Depending on the scanning method and/or scan settings selected, which for example are optimized to detect bone density, it may happen that the bite register is only partially included or recorded, if at all, i.e. that the bite register is entirely or at least partially transparent for the scanning method in question.

The scanning method used to record the structure data of the jaw is, for example, computed tomography (CT), digital volumetric tomography (DVT), or magnetic resonance imaging (MRI). In the course of computed tomography, for example, a plurality of X-ray images taken from different directions are evaluated in a computer-aided manner in order to create an overlap-free, two-dimensional or three-dimensional representation of the bone structure.

By way of example, the results of the scanning method are used to record the exact bone structure of the jaws for subsequent positioning of the implant holes.

By way of example, the jaw or the opposing jaw comprises teeth and/or a dental prosthesis, which are likewise detected using the scanning method and, for example, are likewise transferred to DICOM.

By way of example, the bite register comprises a plurality of opaque markers and/or one or more opaque portions, which are likewise detected using the scanning method and, for example, are likewise transferred to DICOM. Opaque refers to the property whereby the markers and/or portions in question are opaque for the radiation used by the scanning method to record the structure data and therefore are visible in the corresponding scan.

By way of example, there is no need to record the opposing jaw and/or to provide the structure data of the opposing jaw if only one jaw is to be treated and, for example, the opposing jaw has no teeth and/or dental prosthesis. By way of example, there is no need for the structure data of the opposing jaw to be transferred to DICOM. This could have the advantage that the opposing jaw can be disregarded if it is not to be treated and if it additionally has no reference points in the form of teeth or a dental prosthesis. Opaque markers and/or opaque portions of the bite register could be used as reference points.

By way of example, during the scan to record the structure data, a further object which is opaque for the scanning method can be arranged as a reference object in the oral cavity and can be recorded. By way of example, this further object, when arranged in the oral cavity, has a measurable position relative to the bite register and the jawbone.

In block 404, positions for the implant holes and/or fixing holes in the jaw are planned and digitally defined using the 3D model D1 of the jawbone structure. By way of example, the positions of the holes, in particular of the implant holes, are defined such that one or more positioning criteria are satisfied. Here, use is made, for example, of information about the bone structure, in particular the bone wall thickness, the positions of the main nerves and/or the positions of the blood vessels in the jaw. The positioning criteria may include, for example, one or more of the following criteria: extending the hole through a bone wall having a predefined minimum thickness, maintaining a minimum distance of the hole from main nerves in the jaw, and/or maintaining a minimum distance of the hole from blood vessels in the jaw.

By way of example, in order to plan the positions of the holes, the model D1 is also supplemented with visual representations or models of the teeth provided by the dental prosthesis to be produced. By way of example, tooth models from libraries and/or images of the patient's original teeth are used to visualize the teeth.

Alternatively, it is also possible that no visualization of teeth comprised by the dental prosthesis to be produced takes place for planning the positions of the holes.

By way of example, in addition to the implant holes, a plurality of fixing holes is planned in order to fix the drilling template precisely at a predefined position on the patient's gingiva. This could have the advantage that, even though the gingiva provides only a soft support surface for the drilling template, it is still possible to ensure a stable, pressure-independent fixing of the drilling template while the implant holes are being made. This may, for example, enable the patient to open his mouth without the drilling template slipping.

By way of example, the planning of the positioning element and of the drilling template takes place on the basis of the position of the jaws relative to each other during the scan of the bone structures, i.e. the recording position of the jaws. By way of example, the position of the jaws relative to each other that is used for the recording may be of subordinate importance. By way of example, when selecting the distance between the jaws during the recording, it is not necessary to set a predefined distance between the jaws to millimeter accuracy. By way of example, when selecting the dimensions of the bite register and thus the arrangement of the jaws in the recording position, it is possible to disregard how potential teeth provided by a dental prosthesis would stand relative to each other in the position of the jaws selected for the recording. By way of example, it is not necessary that an occlusion with the potential teeth in question is given for the recording position.

By way of example, the planning of the fixing holes takes place for the recording position of the jaws. By way of example, the planning of the implant holes takes place for the recording position of the jaws. By way of example, the dental prosthesis to be fixed in place by the implants is taken into account for the planning of the implant holes.

By way of example, the dimensions of the bite register and thus the arrangement of the jaws in the recording position can be selected such that the jaws are opened far enough to enable the implant holes to be made. If this jaw position is reproduced using the positioning element, this could make it possible for the implant holes to be made in the jaw without removing the positioning element. By way of example, the positioning element has through-openings, through which the implant holes can be made. By way of example, the positioning element has a lattice structure comprising a plurality of support elements, between which such through-openings are arranged.

By way of example, the implant holes used to anchor the implants do not necessarily correspond to one tooth each. By way of example, the positioning element and the drilling template are planned in such a way that the implant holes are made exactly at the planned positions relative to the respective jawbone, each of said positions satisfying one or more positioning criteria. In contrast to this, the positioning relative to the teeth and/or dental prosthesis to be anchored subsequently may, for example, play a subordinate role and/or may be disregarded. Thus, when planning the implant holes, for example, a position priority for secure and stable anchorage could apply, which could be classified higher than a positioning and/or alignment with future teeth to millimeter accuracy.

In block 406, the bite register containing the impression of the gingiva as support surfaces for the template contact surface of the drilling template to be created is scanned using a second scanning method, and a three-dimensional digital representation (D2) of the bite register is created. By way of example, the representation D2 is a three-dimensional digital model of the bite register. By way of example, the second representation D2 comprises the support surfaces for the drilling templates on the gingiva, which defines the template contact surface of the drilling template.

If drilling templates are to be created for both jaws of the patient, the bite register comprises, for example, impressions of the gingivae of both jaws, which each define a support surface for a template contact surface of one of the drilling templates.

By way of example, the second scanning method comprises an optical scan, a computed tomography adjusted to the density of the bite register, and/or a haptic scan.

By way of example, if one of the jaws includes teeth or an existing dental prosthesis, these could be recorded by means of the first scanning method and included in the three-dimensional digital model D1 of the jaw structures. By way of example, these existing teeth and/or the dental prosthesis will be used as reference structures for aligning the three-dimensional digital model D2 of the bite register, based on the second scanning method, relative to the three-dimensional digital model D1 of the jaw, based on the first scanning method, in the same position that the bite register assumed between the jawbones during the recording of the structure data. The relative position and alignment of the bite register relative to the jawbones during the first scanning method can thus be digitally reconstructed in a precise manner. The reference structures in question can be included in the bite register as impressions and thus can also be included in the three-dimensional digital model D2 of the bite register. The reference structures in question, when included in both models D1 and D2, can thus enable the two models to be aligned relative to each other.

By way of example, the two models D1 and D2 can be aligned relative to each other using opaque markers comprised by the bite register. By way of example, the markers in question are opaque, i.e. visible, both for the first scanning method for recording the structure data of the jaw and for the second scanning method for recording the bite register. By way of example, if the markers are visible for both scanning methods, both models D1 and D2 can include the markers in question. By way of example, by bringing the markers of the two models into alignment, the two models D1 and D2 can be aligned relative to each other in the same position that the bite register and the jawbones assumed during the recording of the structure data. The position and alignment of the bite register relative to the jawbones during the first scanning method can thus be digitally reconstructed in a precise manner.

Aligning the two models D1 and D2 relative to each other results, for example, in a combined 3D model which includes the jawbone structure and the surface of the gingiva of at least one jaw, or the surfaces of the gingivae of both jaws if both jaws are being taken into account. In this combined model, the positioning and alignment of the support surface of the gingiva, defined by the bite register, relative to the jawbone corresponds exactly to the actual relative positioning and orientation between the gingival surface and the jawbone during the recording of the structure data, i.e. with the bite register in the patient's mouth.

By way of example, this could have the advantage that an optical scan of the bite register may be sufficient, from which a 3D mesh, point or voxel structure of the support surfaces of the gingiva and/or of the teeth can be created for the drilling template to be produced. By way of example, a complicated CT scan can be omitted if optically detectable reference points are present, such as teeth, a dental prosthesis, or markers.

The mesh is, for example, a polygon mesh, the individual meshes of which have the shape of polygons and which defines the shape of a polyhedron. A polygon mesh comprises vertices, edges, and facets spanned between the edges, which define the shape of a polyhedral object in 3D computer graphics and solid models.

In the case of a dentition equipped with teeth, the 3D model generated from the second scan includes, for example, an exact impression of the antagonists of the opposing jaw, which can be used as reference structures for an exact positioning of the model relative to the jawbone structure. This could have the advantage that, when teeth are used as reference structures, the DICOM format of the first 3D representation D1 can easily be related to the model provided by or extracted from the second 3D representation.

In the case of an entirely toothless dentition, the bite register includes, for example, impressions of the toothless gingivae of both jaws. The bite register is scanned and digitized to create a second 3D model D2. This second 3D model D2 includes, for example, markers of the bite register, which are also included in the first 3D model D1. These markers can be used as reference points and/or reference structures for aligning the two models D1 and D2 if there are no teeth that can be used as reference points. This could have the advantage that, using the markers, the second model D2 can be positioned relative to the first model D1 in a manner corresponding exactly to the recording situation when performing the first scan.

The second model D2 includes, for example, the impressions of the support surfaces of the gingivae that were recorded by the bite register and define the template contact surfaces for the drilling templates to be produced, said impressions having been extracted from the scan data of the second scan. Positioning and aligning the models relative to each other results, for example, in a combined model in which the support surfaces of the gingivae recorded by the bite register are shown in a manner corresponding exactly to the recording situation relative to the jawbones.

By way of example, the markers are opaque both for the first and for the second scanning method. By way of example, the first and the second scanning method are the same scanning methods with different parameter settings. By way of example, both scanning methods are a computed tomography method, which is set for the density of the jawbones in the case of the first scan and for the density of the bite register in the case of the second scan. This could have the advantage that the markers can be detected in both cases. In the second case, the support surfaces of the gingivae, i.e. the surfaces of the bite register, could be precisely recorded at the same time.

By way of example, the first and the second scanning method are different scanning methods. By way of example, the markers are detectable not only for the first scanning method, such as a computed tomography method, a volumetric tomography method or a magnetic resonance imaging method, but also for the second scanning method, for instance an optical scanning method in the optical frequency range or a haptic scanning method.

By way of example, in block 408, the two models D1 and D2 of the jawbones and of the bite register are combined.

The first model D1 of the jaw, for example in the form of a 3D DICOM, is coupled to the second model D2 of the gingival surface, for example in the form of a 3D mesh, point or voxel structure, using the reference points and/or reference structures relative to each other in a manner corresponding to the orientation during the recording of the structure data. These reference points and/or reference structures can be identified and/or selected in the two models D1 and D2, for example manually or automatically by computer, for example using an image recognition algorithm. By way of example, the reference points and/or reference structures are teeth, a dental prosthesis, or markers. By way of example, the markers have a specific geometric shape.

This could have the advantage that the support surfaces of the gingivae can be exactly positioned relative to the jawbones and can be extracted. The extracted support surfaces at the exact positions relative to the jawbones can be used to create a 3D representation which includes the planned holes and the template contact surfaces of the drilling templates in exact relation to each other.

By way of example, in block 410, fixing positions in the support surfaces of the gingiva or gingivae for making fixing holes in the jaw and/or implant positions in the support surfaces of the gingiva or gingivae for making implant holes in the jaw are defined using the combined model.

By way of example, in block 412, the support surfaces of the gingiva or gingivae containing the positions of the planned holes, i.e. fixing holes and/or implant holes, are extracted and one or two drilling templates are created, each comprising a negative of the support surface as a template contact surface and thus being exactly matched to the gingiva in question.

In the drilling template to be created, through-openings in a line, i.e. coaxially, with the planned implant holes are planned at the implant positions. This could have the advantage that a guiding drilling template can be provided for making implant holes for dental implants. Using this drilling template, the positions of the implant holes can be determined exactly.

In addition to the through-openings which define or identify the positions of the fixing holes to be created, the drilling template could comprise, for example, drilling guide elements, by which the corresponding through-openings are provided and which are configured to guide a drilling tool used when making the implant holes and/or to limit the drilling depth of the implant holes.

By way of example, additional through-openings for creating fixing holes, for example laterally arranged fixing holes, can be planned in the drilling template in an analogous manner. In addition to the through-openings which define or identify the positions of the fixing holes to be created, the drilling template could comprise, for example, drilling guide elements, by which the through-openings are provided and which are configured to guide a drilling tool used when making the fixing holes and/or to limit the drilling depth of the fixing holes.

By way of example, in block 414, a positioning element is created which is configured to be arranged between the jaws on the drilling template or between the drilling templates and to provide a pressure-based fixing of the drilling template. When the patient exerts pressure on the positioning element with his jaws, i.e. when he bites on the positioning element, the drilling template(s) are fixed, for example, in each case between the positioning element and a jaw of the patient on which the drilling template is arranged. More specifically, the positioning element may be configured to fix the drilling template in a predefined position on the gingiva, wherein the predefined position corresponds to the position of the bite register during the first scanning method for recording structure data. This could also have the advantage of enabling pressure to be exerted on the drilling template by both jaws.

By way of example, exactly one positioning element is used to fix the drilling template. By way of example, one positioning element is used for each drilling template. By way of example, each positioning element is formed in one piece. By way of example, the positioning elements are each comprised by a drilling template. By way of example, in the case of two drilling templates, the exactly one positioning element is jointly comprised by both drilling templates.

The positioning element could have the advantage of making it easier to position and fix the drilling template in a pressure-based manner. By way of example, the drilling template is kept in correct spatial relation to the jawbone. By way of example, the positioning element can keep the drilling template in correct spatial correlation to the bone of the opposing jaw. By way of example, in the case of two drilling templates, the positioning element can keep the two drilling templates in correct spatial relation to each other.

By way of example, the positioning elements are in each case connected to the drilling templates by means of a non-destructively releasable connection. By way of example, the exactly one positioning element is connected to each of the two drilling templates by a respective non-destructively releasable connection. This could have the advantage that the positioning element can be removed once the fixing holes have been made and the fixing elements have been inserted, and the drilling template is kept in the predefined position on the gingiva by means of the fixing elements. This could provide better access to the through-openings in the drilling template in order to make the implant holes.

This could also have the advantage that relative shifts, in particular lateral shifts, between the positioning element and the drilling template(s) can be prevented.

By way of example, the non-destructively releasable connection between the positioning elements is provided using interlocking structures. By way of example, the interlocking structures form a form fit. By way of example, the interlocking structures form a plug-in connection. By way of example, the positioning element has female parts for receiving male parts of the drilling template, and/or has male parts for being received in female parts of the drilling template. By way of example, the drilling template has female parts for receiving male parts of the positioning element, and/or has male parts for being received in female parts of the positioning element.

By way of example, the positioning element comprises a jaw contact surface for the opposing jaw, the shape of which corresponds to the impression, recorded using the bite register, of the opposing jaw or of gingiva, teeth and/or dental prostheses, such as implants and/or dentures, of the opposing jaw.

By way of example, the combination of positioning element and drilling template(s) comprises two contact surfaces for the two jaws, for instance two template contact surfaces or one template contact surface and one jaw contact surface of the positioning element, the shapes of which correspond to the impressions of the two jaws recorded using the bite register. The relative alignment and the distance between the two contact surfaces corresponds, for example, to the relative alignment and the distance between the two support surfaces of the gingivae recorded as impressions by the bite register.

By way of example, the position and alignment of the jaws during the first scanning method can be exactly reconstructed using the combination of positioning element and drilling template(s). The fixing positions of the fixing holes and/or the implant positions of the implant holes were determined on the basis of the result of this first scanning method. It can thus be ensured that the drilling template and the fixing holes and/or implant holes defined by the drilling template can be positioned exactly as planned. By way of example, by using the fixing holes, the drilling template can be arranged exactly at the planned position on the gingiva in order to make the implant holes exactly at the planned implant positions.

By way of example, in the case of two drilling templates, the two drilling templates each comprise a positioning element, which could have the advantage that the two drilling templates each keep the other in a predetermined position relative to the two jaws. This could also have the advantage that no additional element is required for positioning purposes.

By way of example, in the case of two drilling templates, the two drilling templates each comprise a template contact surface, the shape of which corresponds in each case to one of the impressions of the gingivae recorded using the bite register. By way of example, the relative alignment and the distance between the two template contact surfaces that result from arranging one or two positioning elements between the drilling templates corresponds to the relative alignment and the distance between the two support surfaces recorded as impressions by the bite register.

By way of example, in the case of one drilling template, the drilling template comprises a template contact surface and the positioning element comprises a jaw contact surface, the shape of which corresponds respectively to an impression of the gingiva of the jaw recorded using the bite register or to an impression of the opposing jaw recorded using the bite register. By way of example, the relative alignment and the distance between the template contact surface and the jaw contact surface that result from the use of the positioning element corresponds to the relative alignment and the distance between the two support surfaces recorded as impressions by the bite register.

This could have the advantage that, when using the drilling template in combination with the one or two positioning elements, the jaws are arranged in the recording position and pressure can be exerted on the drilling template by the jaws. By virtue of this pressure, the positioning elements can be temporarily fixed on the jaws, for example in order to make the fixing holes.

By way of example, in the case of two drilling templates which each have a positioning element, the two positioning elements comprise interlocking structures so as to form a non-destructively releasable connection. This could have the advantage of facilitating a relative alignment of the two drilling templates. By means of the positioning elements, the drilling templates could keep each other in correct spatial correlation to the jaws.

Furthermore, by means of the positioning elements, the drilling templates could keep each other in correct spatial relation to each other. This could have the advantage that relative shifts, in particular lateral shifts, between the drilling templates can be prevented.

By way of example, the interlocking structures form a form fit. By way of example, interlocking structures form a plug-in connection. By way of example, the positioning element of a first of the two drilling templates has female parts for receiving male parts of the positioning element of the second drilling template, and/or has male parts for being received in female parts of the positioning element of the second drilling template. By way of example, the positioning element of the second drilling template has female parts for receiving male parts of the positioning element of the first drilling template, and/or has male parts for being received in female parts of the positioning element of the first drilling template.

By way of example, in block 416, the one or two drilling templates and the one or two positioning elements are produced. By way of example, the drilling templates and positioning elements are printed using a 3D printer or are cut from a blank by means of a material-removing machining process.

In block 418, the one or two drilling templates produced and the one or two positioning elements are arranged in a patient's oral cavity and are temporarily fixed by means of pressure, which the patient exerts with his jaws.

By way of example, in block 420, fixing holes are made in one or both jaws of the patient by using the relevant drilling template. Fixing elements are inserted in these fixing holes in order to fix the relevant drilling template, independently of a pressure applied by the jaws, at the predefined positions for which the implant positions were defined.

In block 422, the one or two positioning elements, if these have releasable connections to the drilling templates, are removed from the patient's oral cavity and the implant holes are made at the planned positions in the jaws by using the drilling templates fixed on the jaws by means of the fixing holes.

If the positioning element is comprised by the drilling templates and is not intended to be removed from the patient's oral cavity, the implant holes could also be made without removing the positioning elements. To this end, the positioning elements have, for example, access openings which provide access to the through-openings of the drilling templates in order to make the implant holes. By way of example, the implant holes could in this case also be made without prior fixing by means of fixing elements, i.e. without the step according to block 420, with the drilling templates being fixed in the correct position by means of the pressure of the jaws.

By way of example, the bite register, the drilling template and/or the positioning element comprise a pressure gauge and/or a pressure indicator. This could have the advantage that, during the first scanning method, the pressure applied to the bite register by the patient's jaws is measured and, for example, is adjusted to a predefined value. Furthermore, this pressure applied during the first scanning method could be exactly simulated or reproduced using the positioning element. Therefore, while making the fixing holes, for example, the same pressure of the jaws on the drilling template can be set as when the impressions were taken using the bite register.

By way of example, the bite register and/or the positioning element comprises a material which has pressure-dependent deformation properties and/or color properties, by means of which a pressure exerted on the bite register and/or positioning element can be read.

This could have the advantage of enabling the drilling template to be fixed in a predefined position for making fixing holes, which corresponds exactly to the position for which the bone structure of the jaw was recorded and the drilling template was planned. In particular, the pressure on the soft gingiva could be adjusted exactly to the pressure conditions under which the impressions were taken.

This could have the advantage of enabling the drilling template to be temporarily fixed, in a pressure-based manner, in a position on the soft gingiva of the jaw which corresponds exactly to the position of the bite register on the gingiva during the first scanning method. In this position, for example, the fixing holes can be made.

The pressure-dependent fixing of the drilling template takes place, for example, using an oppositely directed pressure on the drilling template in the direction of the template support surface and the contact surface of the gingiva. This pressure results, for example, from a pressure that is exerted on the drilling template by the two jaws.

The pressure-independent fixing takes place, for example, using fixing elements which are inserted in the fixing holes. By way of example, this pressure-independent fixing enables the jaws to be opened in order to make the implant holes, without the drilling template slipping. This could have the advantage that, in particular, the original position of the bite register when recording the support surface is taken into account. Embodiments can thus have the advantage that incorrect positioning can be avoided.

The position of the original bite register could therefore be simulated or reproduced even though it is no longer the bite register that is being used, but rather the drilling template or drilling templates in combination with one or two positioning elements. Such a simulation of the bite register by using a positioning element may have the effect that, not only do the angles of the support surface of the drilling template relative to the jawbone correspond exactly to the angles of the support surface relative to the jawbone recorded using the bite register, but also the magnitude and direction of the pressure on the soft tissue of the gingiva for the pressure-dependent fixing of the drilling template while making the fixing holes can be set or reproduced so as to be identical or at least very similar to the pressure with which the impression of the bite register was taken during the first scanning method.

This could have the advantage that deformations of the soft tissue of the jaws, e.g. of the gingiva, while making the fixing holes can be reproduced in a manner identical or at least very similar to deformations during the first scanning method and during the creation of the bite register.

This could have the advantage of enabling easy and precise reproduction of spatial conditions of the jaw under which the impressions were taken using the bite register and the first scanning method was carried out.

This could have the advantage that only a small number of elements, for example one, two, three or four elements, are necessary for this, i.e. for example one to two drilling templates and one to two positioning elements, wherein the positioning elements may be comprised by the drilling templates or may be releasably connected thereto.

These elements, in combination with each other, simulate the bite register. It is possible to simulate the same pressure and position conditions under which the bite register was created and the first scanning method for recording the structure data was carried out. This could have the advantage that the fixing holes for fixing the drilling template on the patient's jaw can be made with high precision at the planned position on the patient's jaw.

### LIST OF REFERENCE SIGNS

10 computer system
11 production device
24 screen
50 user interface
52 control element
54 hardware component
56 external device
58 external device
60 3D printer
62 printing element
64 machining device
65 machining tool
66 holding device
67 blank
68 material
70 scanning device
72 scanning device
120 jawbone
121 opposing jawbone
122 gingiva
123 gingiva
124 gingival surface
125 gingival surface
126 dental object
130 bite register
132 impression
133 impression
134 marker
136 pressure sensing element
137 pressure indicating element
140 scan data of jawbone
141 scan data of opposing jawbone
142 scan data of marker
144 scan data of dental object
150 scan data of bite register
152 scan data of marker
154 scan data of impression
155 scan data of impression
160 digital model of jaw/bite register
170 digital model of positioning element/drilling template
200 positioning element
201 positioning element
202 contact surface
203 contact surface
204 connecting element
205 connecting element
206 connecting element
207 connecting element
208 connecting element
209 connecting element
210 access opening
211 access opening
212 contact surface
214 connecting element
216 pressure sensing element
217 pressure indicating element
218 contact surface
219 contact surface
220 drilling template
221 drilling template
222 template contact surface
223 template contact surface
224 through-opening
225 through-opening
226 through-opening
227 through-opening
228 connecting element
229 connecting element
230 drilling template surface
231 drilling template surface

1. A method for producing a first positioning element for fixing a first drilling template on a gingiva of a patient's jaw in a first position relative to a jawbone of the jaw,
wherein the first positioning element is configured to fix the first drilling template, by means of pressure, which the patient applies to the first positioning element by closing the jaw and an opposing jaw, in the first position for making first implant holes in the jaw for first dental implants,
wherein the first drilling template comprises a first template contact surface for making contact with a surface of the gingiva of the jaw and comprises first through-openings, wherein the first through-openings each define a first implant position for making one of the first implant holes,
wherein the method comprises:
recording first shape data of a first impression of the surface of the gingiva of the jaw, using a bite register arranged between the patient's two jaws, wherein the two jaws are located in a recording position relative to each other, wherein the first impression is located in the first position relative to the jawbone, and wherein the first impression defines the first template contact surface,
recording first structure data of the jawbone while the bite register is arranged between the two jaws in the recording position,
recording position data of the bite register relative to the jawbone while the bite register is arranged between the two jaws in the recording position,
creating a first three-dimensional digital model, using the recorded first shape data, first structure data and position data, wherein the first model comprises the jawbone structure and the surface of the gingiva of the jaw,
defining the first implant positions for making the first implant holes in the jaw, using the first model,
creating a second three-dimensional digital model of the first drilling template and of the first positioning element, using the first model and the defined first implant positions, wherein the shape and dimensions of the first positioning element are determined such that, when pressure is applied to the first positioning element, the two jaws are arranged in the recording position and the first drilling template is arranged in the first position relative to the jawbone,
producing the first positioning element, using the second three-dimensional digital model.

2. (canceled)

3. The method according to claim 1, wherein the first positioning element is configured to fix the first drilling template in the first position for making first fixing holes in the jaw for first fixing elements,
wherein the first drilling template further comprises second through-openings, wherein the second through-openings each define a first fixing position for making one of the first fixing holes, wherein the first fixing elements are configured to fix the first drilling template, in a jaw pressure-independent manner, in the first position for making the first implant holes in the jaw for the first dental implants,
wherein the method further comprises defining the first fixing positions for making the first fixing holes in the jaw, using the first model, and the second three-dimensional digital model of the first drilling template and of the first positioning element is also created using defined first fixing positions.

4. The method according to claim 1, wherein the method further comprises producing the first drilling template, using the second three-dimensional digital model.

5. The method according to claim 1, wherein the first positioning element comprises one or more first connecting elements, which are configured to establish and to release a non-destructively releasable first connection between the first positioning element and the first drilling template in the patient's oral cavity.

6. (canceled)

7. The method according to claim 1, wherein second shape data of a second impression of a surface of the opposing jaw are recorded, using the bite register arranged between the two jaws in the recording position, said second shape data being used to create the first three-dimensional digital model, which further comprises the second surface of the opposing jaw.

8. (canceled)

9. The method according to claim 7, wherein the method further comprises recording second structure data of an opposing jawbone of the opposing jaw while the bite register is arranged between the two jaws in the recording position,
wherein the second structure data are used to create the first three-dimensional digital model, which further comprises the opposing jawbone structure,
wherein the method further comprises producing a second drilling template which comprises a second template contact surface for making contact with the surface of the opposing jaw, wherein the second impression defines the second template contact surface, wherein the surface of the opposing jaw is a surface of the gingiva of the opposing jaw,
wherein the second drilling template further comprises third through-openings, wherein the third through-openings each define a second implant position for making a second implant hole in the opposing jaw for second dental implants,
wherein the recorded position data comprise position data of the bite register relative to the opposing jawbone,
wherein the method further comprises defining the second implant positions for inserting the second implants into the opposing jaw, using the first model,
wherein the second model comprises the second drilling template, and wherein the defined second implant positions are also used to create the second model.

10. (canceled)

11. The method according to claim 9, wherein the first positioning element is further configured to fix the second drilling template in the second position by means of pressure, which the patient applies to the first positioning element by closing the two jaws, wherein the shape and dimensions of the first positioning element are further determined such that the second drilling template is arranged in the second position relative to the opposing jaw.

12. The method according to claim 9, wherein the first positioning element is configured to fix the second drilling template in the second position for making second fixing holes in the opposing jaw for second fixing elements,
wherein the second drilling template further comprises fourth through-openings, wherein the fourth through-openings each define a second fixing position for making one of the second fixing holes, wherein the second fixing elements are configured to fix the second drilling template, in a jaw pressure-independent manner, in a second position relative to the opposing jawbone for making the second implant holes in the opposing jaw for the second dental implants,
wherein the method further comprises defining the second fixing positions for inserting the second fixing elements in the opposing jaw, using the first model,
wherein the defined second fixing positions are also used to create the second model.

13. The method according to claim 12, wherein the first positioning element is further configured to fix the second drilling template in the second position for making the second fixing holes in the opposing jaw for the second fixing elements.

14. (canceled)

15. (canceled)

16. (canceled)

17. The method according to claim 9, wherein the production of the second drilling template further comprises producing a second positioning element, wherein the second positioning element in combination with the first positioning element is configured to fix the second drilling template in the second position by means of pressure, which the patient applies to the first and second positioning element by closing the jaws, wherein the shape and dimensions of the second positioning element are determined such that, when the pressure is applied to the first and second positioning element, the two jaws are arranged in the recording position and the second drilling template is arranged in the second position relative to the opposing jaw.

18. The method according to claim 17, wherein the second positioning element comprises one or more third connecting elements, which are configured to establish and to release a non-destructively releasable third connection between the second positioning element and the second drilling template in the patient's oral cavity.

19. (canceled)

20. (canceled)

21. The method according to claim 9, wherein the second implant positions of the second implant holes and/or second fixing positions of the second fixing holes are each defined in such a way as to satisfy one or more of the following first positioning criteria: extending the second implant holes and/or second fixing holes through a bone wall of the opposing jawbone structure having a predefined first minimum thickness, maintaining a predefined first minimum distance of the second implant holes and/or second fixing holes from main nerves in the opposing jaw, maintaining a predefined second minimum distance of the second implant holes and/or second fixing holes from blood vessels in the opposing jaw.

22. (canceled)

23. (canceled)

24. The method according to claim 1, wherein the first implant positions of the first implant holes and/or first fixing positions of the first fixing holes are each defined in such a way as to satisfy one or more of the following second positioning criteria: extending the first implant holes and/or first fixing holes through a bone wall of the jawbone structure having a predefined second minimum thickness, maintaining a predefined third minimum distance of the first implant holes and/or first fixing holes from main nerves in the jaw, maintaining a predefined fourth minimum distance of the first implant holes and/or first fixing holes from blood vessels in the jaw.

25. (canceled)

26. (canceled)

27. The method according to claim 1, wherein the first and/or second structure data are recorded using a first scanning method, which is one of the following methods: a computed tomography method, a volumetric tomography method or a magnetic resonance imaging method.

28. The method according to claim 27, wherein the position data of the bite register are recorded using markers, which are comprised by the bite register and are opaque for the first scanning method.

29. The method according to claim 27, wherein the position data of the bite register are recorded using contact between the second impression and a dental object of the opposing jaw which is opaque for the first scanning method.

30. (canceled)

31. The method according to claim 1, wherein the method further comprises sensing a grip pressure applied to the bite register arranged between the patient's two jaws,
wherein the first positioning element and/or the second positioning element comprises a pressure sensing element for checking whether the pressure applied to the first positioning element and/or the second positioning element in the recording position of the two jaws matches the grip pressure.

32. A positioning element for fixing a first drilling template on a gingiva of a patient's jaw in a first position relative to a jawbone of the jaw,
wherein the positioning element is configured to fix the first drilling template, by means of pressure, which the patient applies to the positioning element by closing the jaw and an opposing jaw, in the first position for making first implant holes in the jaw for first dental implants,
wherein the positioning element is produced using a method which comprises:
recording first shape data of a first impression of the surface of the gingiva of the jaw, using a bite register arranged between the patient's two jaws, wherein the two jaws are located in a recording position relative to each other, wherein the first impression is located in the first position relative to the jawbone, and wherein the first impression defines a first template contact surface of a first drilling template for making contact with a surface of the gingiva of the jaw,
recording first structure data of the jawbone while the bite register is arranged between the two jaws in the recording position,
recording position data of the bite register relative to the jawbone while the bite register is arranged between the two jaws in the recording position,
creating a first three-dimensional digital model, using the recorded first shape data, first structure data and position data, wherein the first model comprises the jawbone structure and the surface of the gingiva of the jaw,
defining first implant positions for making the first implant holes in the jaw, using the first model,
creating a second three-dimensional digital model of the first drilling template and of the positioning element, using the first model and the defined first implant positions, wherein the shape and dimensions of the positioning element are determined such that, when pressure is applied to the positioning element, the two jaws are arranged in the recording position and the first drilling template is arranged in the first position relative to the jawbone, wherein the first drilling template comprises the first template contact surface and first through-openings, wherein the first through-openings each define one of the first implant positions,
wherein the second three-dimensional digital model is used to produce the positioning element.

33. (canceled)

34. A combination of the positioning element according to claim 32 and a first drilling template produced using the second three-dimensional digital model.

35. (canceled)

36. (canceled)

37. (canceled)

38. The combination according to claim 34, further comprising a second drilling template which comprises a second template contact surface for making contact with the surface of the opposing jaw, wherein the second impression defines the second template contact surface, wherein the surface of the opposing jaw is a surface of the gingiva of the opposing jaw,
wherein the second drilling template further comprises third through-openings, wherein the third through-openings each define a second implant position for making one of the second implant holes.

39. (canceled)

40. (canceled)

41. (canceled)

42. (canceled)

43. (canceled)

44. (canceled)

45. (canceled)

46. (canceled)

47. (canceled)

48. (canceled)
